(19) United States
(12) Patent Application Publication
(10) Pub. No.: US 2026/0076741 A1
(43) Pub. Date: Mar. 19, 2026

(54) LASER BLADE PULSED LASER ABLATION

(71) Applicants: Smith & Nephew, Inc., Memphis, TN (US); Smith & Nephew Asia Pacific Pte. Limited, Singapore (SG); Smith & Nephew Orthopaedics AG, Zug (CH)

(72) Inventors: Darren J. WILSON, Hull (GB); Brett J. BELL, Mendon, UT (US)

(21) Appl. No.: 19/399,901

(22) Filed: Nov. 25, 2025

Related U.S. Application Data
(63) Continuation-in-part of application No. 18/662,181, filed on May 13, 2024.
(60) Provisional application No. 63/466,140, filed on May 12, 2023.

(51) Int. Cl. A61B 18/22; A61B 18/00; A61B 18/20; A61B 34/30

(52) U.S. Cl. CPC A61B 18/22; A61B 34/30; A61B 2018/00601; A61B 2018/00648; A61B 2018/2015; A61B 2018/2211; A61B 2018/2222

(57) ABSTRACT

A device for resecting hard biological tissue is provided. The device comprises a laser source configured to emit a laser beam, and a plurality of optical fibers, each having a proximal end optically coupled to the laser source and a distal end configured to emit the laser beam. A support structure maintains the distal ends of the optical fibers in a predetermined spatial arrangement. A window is positioned to permit transmission of the laser beam from the distal ends toward the tissue. At least one spacing element is associated with the support structure to maintain a predetermined distance between the distal ends and the tissue during operation. The device further includes a fluid delivery system associated with the support structure and configured to deliver a fluid to a cutting interface at the tissue. The arrangement enables precise, efficient, and controlled laser ablation or resection of hard tissue, with improved cooling and debris management.

### CROSS REFERENCE TO RELATED APPLICATIONS

The present application is a continuation-in-part of, claims priority to, and the benefit of, U.S. Nonprovisional application Ser. No. 18/662,181, titled “ROBOTICALLY CONTROLLED LASER-ASSISTED HANDPIECE,” filed May 13, 2024, which claims priority to, and the benefit of, U.S. Provisional Application No. 63/466,140, titled “ROBOTICALLY CONTROLLED LASER-ASSISTED HANDPIECE,” filed May 12, 2023, the contents of both are hereby incorporated by reference in their entireties.

### TECHNICAL FIELD

The present disclosure relates generally to methods, systems, and apparatuses related to surgical resection. More particularly, the present disclosure relates to a hand-held, semi-autonomous robotic resection tool, which the surgeon can move freely in space.

### BACKGROUND

Bone tissue is a complex, anisotropic, and brittle material that presents significant challenges during surgical resection. The primary requirements for bone resection procedures include high efficiency, precise accuracy, and minimal thermal and mechanical damage to the surrounding tissue. Achieving these requirements is complicated by the inherent properties of bone, which can lead to complications such as thermal necrosis, microcracking, and compromised healing at the surgical site.

Conventional mechanical instruments, such as oscillating saws and rotary burrs, are widely used for bone cutting in orthopedic, dental, and craniofacial surgeries. While these tools are capable of removing bone at clinically acceptable rates, they are associated with several drawbacks. Mechanical cutting can result in irregular crack propagation, the formation of bone chips, and the generation of significant heat at the cutting interface. Elevated temperatures during resection can cause tissue necrosis and delay postoperative healing. Additionally, the mechanical action of these instruments produces bone debris, which may impede bone regeneration and present a biohazard risk if aerosolized in the surgical environment.

A persistent problem in the field of bone surgery is the difficulty of achieving efficient and accurate bone removal while minimizing adverse effects such as excessive heat generation, mechanical trauma, and the production of debris. These challenges can negatively impact surgical outcomes, prolong recovery times, and increase the risk of complications for both patients and surgical staff.

### SUMMARY

In some aspects, the techniques described herein relate to an apparatus for resecting hard biological tissue, including: a laser source configured to emit a laser beam; a plurality of optical fibers, each optical fiber having a proximal end optically coupled to the laser source and a distal end configured to emit the laser beam from the laser source; a support structure configured to maintain the distal ends of the plurality of optical fibers in a predetermined spatial arrangement; a window positioned configured to permit transmission of the laser beam from the distal ends of the plurality of optical fibers toward the tissue; at least one spacing element associated with the support structure and configured to maintain a predetermined distance between the distal ends of the plurality of optical fibers and the tissue during operation; and a fluid delivery system associated with the support structure and configured to deliver a fluid to a cutting interface associated with the tissue.

In some aspects, the techniques described herein relate to an apparatus, wherein: the plurality of optical fibers are arranged in a linear or hexagonal array at the distal ends of the plurality of optical fibers.

In some aspects, the techniques described herein relate to an apparatus, wherein: the laser source is further configured to emit the laser beam having a substantially uniform intensity profile at the distal ends of the plurality of optical fibers.

In some aspects, the techniques described herein relate to an apparatus, wherein: the window includes a material selected from the group consisting of sapphire and glass.

In some aspects, the techniques described herein relate to an apparatus, wherein: the at least one spacing element includes one or more projections extending from the support structure and configured to contact the tissue and to maintain the predetermined distance.

In some aspects, the techniques described herein relate to an apparatus, wherein: the fluid delivery system includes at least one nozzle configured to deliver a mist of fluid to the cutting interface.

In some aspects, the techniques described herein relate to an apparatus, wherein: the fluid delivery system further includes a suction channel configured to evacuate excess fluid and ablation byproducts from the cutting interface.

In some aspects, the techniques described herein relate to an apparatus, further including: a non-contact sensor associated with the support structure and configured to provide real-time feedback regarding at least one of temperature, ablation depth, or tissue differentiation at the cutting interface.

In some aspects, the techniques described herein relate to an apparatus, wherein: the sensor includes an optical coherence tomography (OCT) sensor configured to measure ablation depth.

In some aspects, the techniques described herein relate to an apparatus. According to an implementation, confocal sensors may be used to measure distance via wavelength. According to an implementation, these sensors may focus a specific wavelength of light on the surface and measure the returned light's spectrum to determine height. This method may be highly accurate and effective on transparent, opaque, and highly reflective surfaces, such as bone.

In some aspects, the techniques described herein relate to an apparatus wherein a laser triangulation sensor is used. According to an implementation, the laser triangulation sensor may emit a laser beam onto the surface of the bone. A camera or receiver captures the reflection at an angle. The sensor uses the principle of triangulation to calculate the distance and create a 3D height map of the surface, ensuring the correct focal point is maintained for consistent cutting depth.

In some aspects, the techniques described herein relate to an apparatus, further including: one or more processors configured to receive feedback from the sensor and to control at least one of the laser source, the fluid delivery system, or the support structure based on the feedback.

In some aspects, the techniques described herein relate to an apparatus, wherein: the support structure includes a disposable outer portion and a reusable inner portion containing the plurality of optical fibers.

In some aspects, the techniques described herein relate to an apparatus, wherein: the apparatus is configured for use with a robotic guidance system or a hand-held operation mode.

In some aspects, the techniques described herein relate to an apparatus, wherein: the laser source is configured to emit at a wavelength between 2.7 and 3.1 microns.

In some aspects, the techniques described herein relate to an apparatus, further including: an actuator configured to oscillate the support structure to increase the effective width of the resection at the cutting interface.

In some aspects, the techniques described herein relate to an apparatus, wherein: the actuator includes a piezoelectric transducer configured to impart linear or ultrasonic oscillation to the support structure.

In some aspects, the techniques described herein relate to an apparatus for resecting hard biological tissue, including: a laser source configured to emit a laser beam; a plurality of optical fibers, each optical fiber having a proximal end optically coupled to the laser source and a distal end configured to emit the laser beam from the laser source; a support structure configured to maintain the distal ends of the plurality of optical fibers in a predetermined spatial arrangement; a protective transparent window positioned configured to permit transmission of the laser beam from the distal ends of the plurality of optical fibers toward the tissue; at least one spacing element associated with the support structure and configured to maintain a predetermined distance between the distal ends of the plurality of optical fibers and the tissue during operation; a fluid delivery system associated with the support structure and configured to deliver a fluid to a cutting interface associated with the tissue; and a suction channel integrated with the support structure and configured to evacuate excess fluid and ablation byproducts from the cutting interface.

In some aspects, the techniques described herein relate to an apparatus, further including: a sensor including an optical coherence tomography (OCT) sensor configured to measure ablation depth at the cutting interface.

In some aspects, the techniques described herein relate to a method for resecting hard biological tissue, including: emitting a laser beam from a laser source; transmitting the laser beam through a plurality of optical fibers, each optical fiber having a proximal end optically coupled to the laser source and a distal end configured to emit the laser beam from the laser source; maintaining the distal ends of the plurality of optical fibers in a predetermined spatial arrangement using a support structure; positioning a window to permit transmission of in the laser beam from the distal ends of the plurality of optical fibers toward the tissue; maintaining a predetermined distance between the distal ends of the plurality of optical fibers and the tissue using at least one spacing element associated with the support structure; delivering a fluid to a cutting interface using a fluid delivery system associated with the support structure; and resecting the tissue by moving the cutting interface relative to the tissue while emitting the laser beam.

In some aspects, the techniques described herein relate to a method, further including: arranging the plurality of optical fibers in a linear or hexagonal array at the distal ends of the plurality of optical fibers.

In some aspects, the techniques described herein relate to a method, further including: evacuating excess fluid and ablation byproducts from the cutting interface using a suction channel integrated with the support structure.

### BRIEF DESCRIPTION OF THE DRAWINGS

The accompanying drawings, which are incorporated in and form a part of the specification, illustrate the embodiments of the invention and together with the written description serve to explain the principles, characteristics, and features of the invention. In the drawings:

FIG. 1 depicts an operating theatre including an illustrative computer-assisted surgical system (CASS) in accordance with an embodiment.

FIG. 2A depicts illustrative control instructions that a surgical computer provides to other components of a CASS in accordance with an embodiment.

FIG. 2B depicts illustrative control instructions that components of a CASS provide to a surgical computer in accordance with an embodiment.

FIG. 2C depicts an illustrative implementation in which a surgical computer is connected to a surgical data server via a network in accordance with an embodiment.

FIG. 3 illustrates seven bone cuts performed in a typical total knee arthroplasty (TKA) procedure in accordance with an embodiment.

FIG. 4 illustrates a graph detailing spot diameters and associated details relating to Gaussian and non-Gaussian/tophat beam approaches in accordance with an embodiment.

FIG. 5 illustrates a schematic of a single laser spot with beam shaping and refined optics for a top-hat beam shape in accordance with an embodiment.

FIG. 6 depicts a perspective view of the laser blade apparatus in accordance with an embodiment.

FIG. 7 illustrates a perspective view of the laser blade apparatus in a treatment device in accordance with an embodiment.

FIG. 8A illustrates a perspective view of the sapphire bundle of the laser blade apparatus in accordance with an embodiment.

FIG. 8B illustrates a front view of the sapphire bundle of the laser blade apparatus in accordance with an embodiment.

FIG. 9A illustrates a perspective image view of the laser blade apparatus in accordance with an embodiment.

FIG. 9B illustrates a front view of the coupling facet of the laser blade apparatus in accordance with an embodiment.

FIG. 10A illustrates a perspective image view of the outer housing of the laser blade apparatus in accordance with an embodiment.

FIG. 10B illustrates a top-front view of the outer housing of the laser blade apparatus, detailing the inner window, in accordance with an embodiment.

FIG. 10C illustrates a perspective isolated view of the integrated window of the outer housing of the laser blade apparatus in accordance with an embodiment.

FIG. 11 illustrates a perspective view of the fiber cartridge end facet collimation of the laser blade apparatus in accordance with an embodiment.

FIG. 12A illustrates a perspective image view of the laser blade apparatus in accordance with an embodiment.

FIGS. 12B-12E illustrate additional perspective image views of the laser blade apparatus in accordance with an embodiment.

FIG. 13 illustrates a schematic workflow of the top-hat ER laser optical system incorporating the laser blade apparatus in accordance with an embodiment.

FIG. 14A illustrates an image view of the schematic workflow of the top-hat ER laser optical system incorporating the laser blade apparatus, without an optical fiber, in accordance with an embodiment.

FIG. 14B illustrates an image view of the schematic workflow of the top-hat ER laser optical system incorporating the laser blade apparatus, with an optical fiber, in accordance with an embodiment.

FIG. 15 illustrates a perspective view of a laser blade apparatus supported by piezoelectric ultrasonics for vibration-assisted laser cutting in accordance with an embodiment.

FIG. 16 illustrates is a flowchart of an example method for resecting biological tissue in accordance with an embodiment.

It should be understood that the drawings are not necessarily to scale and that the embodiments are sometimes illustrated by graphic symbols, phantom lines, diagrammatic representations, and fragmentary views. In certain instances, details that are not necessary for an understanding of the invention or that render other details difficult to perceive may have been omitted.

### DETAILED DESCRIPTION

This disclosure is not limited to the particular systems, devices and methods described, as these may vary. The terminology used in the description is for the purpose of describing the particular versions or embodiments only and is not intended to limit the scope.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

The present disclosure includes a system and method for bone ablation utilizing a laser energy source configured to deliver energy to bone tissue for resection, shaping, or removal. The system is compatible with a wide range of laser wavelengths, pulse durations, and energy profiles, and is not restricted to any particular type of laser or emission mode. The present disclosure includes the use of beam shaping optics or elements to modify the spatial energy distribution of the laser, such as to achieve a uniform, top-hat, or otherwise optimized intensity profile at the target site. Laser energy may be delivered to the bone through one or more optical fibers, which can be arranged in various configurations—including linear, circular, hexagonal, or custom arrays—and may be bundled, multiplexed, or otherwise combined to achieve the desired ablation characteristics. The distal end of the fiber or fiber bundle may be integrated into an end effector, which can take the form of a blade, probe, cannula, or other structure suitable for surgical manipulation, and may include features such as feet, projections, spacers, or guides to maintain a fixed or controlled distance from the bone surface. According to another aspect of the present disclosure, the end effector design may be tailored for different beam cutting patterns. In this way, other non-linear arrangements of the laser fibers may be configured to achieve novel optical effects and create new types of end-effectors. More specifically, a non-linear approach can be used to control a laser beam's shape and/or intensity. For example, an end-effector with a linear arrangement of laser fibers produces a uniform, elongated cutting profile, while a circular arrangement creates a concentrated, central cutting spot. According to an implementation, this choice may depend on the specific cutting application and desired outcome (including the shape of the cut), the material's properties, and the required cutting speed. For example, in a linear fiber arrangement, a multi-fiber array or swept single fiber may be included as an end effector type. The beam shape and profile may include an elongated or linear beam profile, which can be uniform or scanned back and forth. The cut shape may be in a straight line, with cutting action suitable for making straight-line narrow incisions and/or creating long, shallow cuts. The energy distribution may distribute energy over a larger, rectangular area, which results in a overlapping bean intensity profile. Applications may include resection of large areas of tissue where a long, uniform cut is needed, e.g. planar cuts during joint resurfacing and/or osteotomies. Integration with robotic systems, such as system 100, may include a linear pattern guided with high precision by robotic arms, allowing for accurate, pre-planned cuts.

Further, in a circular fiber arrangement, a single, focused fiber or groups of fibers may be included as an end effector type. The beam shape and profile may produce a circular, highly concentrated laser spot at its focal point. The cut shape may include a spot, hole, or complex shape via motion. Cutting action may include focused ablation to deliver very high power density to a small, concentrated area. The energy distribution may deliver very high power density to a small, concentrated area. Applications may include: precise, small circular holes may be created for drilling holes; targeted and deep tissue ablation be occur with restricted access (intra-articular lesions); a single, small-diameter fiber or group of fibers being delivered through the working channel of an endoscope (making it ideal for minimally invasive procedures); and/or circular-firing tips or spiralling ablation patterns, useful for creating cylindrical boreholes in bone or for reaming applications. Similarly, integration with robotic systems, such as system 100, may be used to create lugs holes for facilitating attachment of implants.

The present disclosure further includes the use of cooling, irrigation, or debris management systems, such as the delivery of a mist, spray, jet, or flow of fluid—including saline, water, air, liquid nitrogen or gas mixtures—to the ablation site, either continuously or in a pulsed or sequenced manner. The system may incorporate one or more suction or evacuation channels to remove excess fluid, debris, or ablation byproducts, helping to maintain a clear optical path for the laser energy. Real-time feedback and control mechanisms, such as temperature sensors, optical coherence tomography (OCT), optoacoustic sensors, or spectroscopic analysis, may be used to monitor and regulate ablation depth, tissue type, or thermal effects, and to adjust system parameters as needed. The system can be operated manually, robotically, or in a hybrid mode, and may be integrated with surgical navigation, tracking, or planning platforms. Modular or disposable components, such as a reusable fiber cartridge, a disposable outer housing combined with a sterile drape, may be included to facilitate sterilization or single-use applications. The handpiece can offer intra-operative planning and depth control with functional laser cut geometries, and robotic assistance for a precise execution, and repeatability of movements, which may be associated with guide-free cutting. The robotically controlled, free-hand resection tool can integrate one or more portable ablation laser (e.g., a compact, diode pumped solid-state laser with an additional co-axially bundled aiming beam) or be tethered to an external laser.

The present disclosure is applicable to a variety of surgical procedures and anatomical sites, including orthopedic, spinal, craniofacial, dental, trauma, or minimally invasive contexts where precise, efficient, and controlled removal or modification of bone or hard tissue is required. The system may be configured for planar, non-planar, curved, or custom-shaped cuts, and can include features for reciprocating, oscillating, or otherwise moving the end effector to achieve the desired resection geometry. Any combination or subcombination of the features described herein is contemplated, encompassing all variations, alternatives, and equivalents that achieve efficient, safe, and precise bone ablation.

### Definitions

For the purposes of this disclosure, the term “implant” is used to refer to a prosthetic device or structure manufactured to replace or enhance a biological structure. For example, in a total hip replacement procedure a prosthetic acetabular cup (implant) is used to replace or enhance a patient's worn or damaged acetabulum. While the term “implant” is generally considered to denote a man-made structure (as contrasted with a transplant), for the purposes of this specification an implant can include a biological tissue or material transplanted to replace or enhance a biological structure.

For the purposes of this disclosure, the term “real-time” is used to refer to calculations or operations performed on-the-fly as events occur or input is received by the operable system. However, the use of the term “real-time” is not intended to preclude operations that cause some latency between input and response, so long as the latency is an unintended consequence induced by the performance characteristics of the machine.

Although much of this disclosure refers to surgeons or other medical professionals by specific job title or role, nothing in this disclosure is intended to be limited to a specific job title or function. Surgeons or medical professionals can include any doctor, nurse, medical professional, or technician. Any of these terms or job titles can be used interchangeably with the user of the systems disclosed herein unless otherwise explicitly demarcated. For example, a reference to a surgeon also could apply, in some embodiments to a technician or nurse.

The systems, methods, and devices disclosed herein are particularly well adapted for surgical procedures that utilize surgical navigation systems, such as the CORI® surgical navigation system. CORI® is a registered trademark of BLUE BELT TECHNOLOGIES, INC. of Pittsburgh, PA, which is a subsidiary of SMITH & NEPHEW, INC. of Memphis, TN.

### CASS Ecosystem Overview

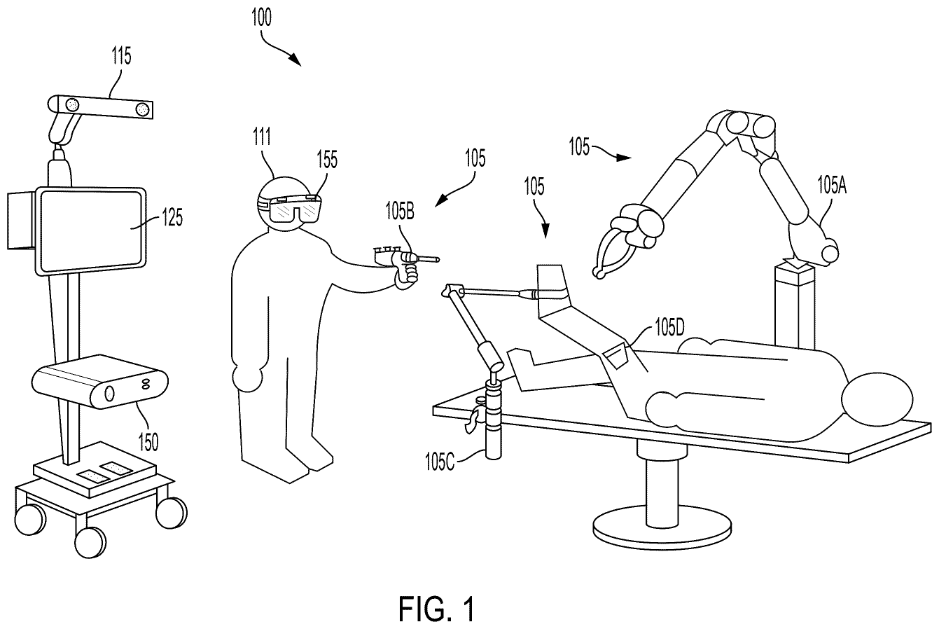

FIG. 1 provides an illustration of an example computer-assisted surgical system (CASS) 100, according to some embodiments. As described in further detail in the sections that follow, the CASS uses computers, robotics, and imaging technology to aid surgeons in performing orthopedic surgery procedures such as total knee arthroplasty (TKA) or THA. For example, surgical navigation systems can aid surgeons in locating patient anatomical structures, guiding surgical instruments, and implanting medical devices with a high degree of accuracy. Surgical navigation systems such as the CASS 100 often employ various forms of computing technology to perform a wide variety of standard and minimally invasive surgical procedures and techniques. Moreover, these systems allow surgeons to more accurately plan, track and navigate the placement of instruments and implants relative to the body of a patient, as well as conduct pre-operative and intra-operative body imaging.

An effector platform 105 positions surgical tools relative to a patient during surgery. The exact components of the effector platform 105 will vary, depending on the embodiment employed. For example, for a knee surgery, the effector platform 105 may include an end effector 105B that holds surgical tools or instruments during their use. The end effector 105B may be a handheld device or instrument used by the surgeon (e.g., a CORI® hand piece or a cutting guide or jig) or, alternatively, the end effector 105B can include a device or instrument held or positioned by a robotic arm 105A. While one robotic arm 105A is illustrated in FIG. 1, in some embodiments there may be multiple devices. As examples, there may be one robotic arm 105A on each side of an operating table T or two devices on one side of the table T. The robotic arm 105A may be mounted directly to the table T, be located next to the table T on a floor platform (not shown), mounted on a floor-to-ceiling pole, or mounted on a wall or ceiling of an operating room. The floor platform may be fixed or moveable. In one particular embodiment, the robotic arm 105A is mounted on a floor-to-ceiling pole located between the patient's legs or feet. In some embodiments, the end effector 105B may include a suture holder or a stapler to assist in closing wounds. Further, in the case of two robotic arms 105A, the surgical computer 150 can drive the robotic arms 105A to work together to suture the wound at closure. Alternatively, the surgical computer 150 can drive one or more robotic arms 105A to staple the wound at closure.

The effector platform 105 can include a limb positioner 105C for positioning the patient's limbs during surgery. One example of a limb positioner 105C is the SMITH AND NEPHEW SPIDER2 system. The limb positioner 105C may be operated manually by the surgeon or alternatively change limb positions based on instructions received from the surgical computer 150 (described below). While one limb positioner 105C is illustrated in FIG. 1, in some embodiments there may be multiple devices. As examples, there may be one limb positioner 105C on each side of the operating table T or two devices on one side of the table T. The limb positioner 105C may be mounted directly to the table T, be located next to the table T on a floor platform (not shown), mounted on a pole, or mounted on a wall or ceiling of an operating room. In some embodiments, the limb positioner 105C can be used in non-conventional ways, such as a retractor or specific bone holder. The limb positioner 105C may include, as examples, an ankle boot, a soft tissue clamp, a bone clamp, or a soft-tissue retractor spoon, such as a hooked, curved, or angled blade. In some embodiments, the limb positioner 105C may include a suture holder to assist in closing wounds.

The effector platform 105 may include tools, such as a screwdriver, light or laser, to indicate an axis or plane, bubble level, pin driver, pin puller, plane checker, pointer, finger, or some combination thereof.

Resection equipment 110 (not shown in FIG. 1) performs bone or tissue resection using, for example, mechanical, ultrasonic, or laser techniques. Examples of resection equipment 110 include drilling devices, burring devices, oscillatory sawing devices, vibratory impaction devices, reamers, ultrasonic bone cutting devices, radio frequency ablation devices, reciprocating devices (such as a rasp or broach), and laser ablation systems. In some embodiments, the resection equipment 110 is held and operated by the surgeon during surgery. In other embodiments, the effector platform 105 may be used to hold the resection equipment 110 during use.

The effector platform 105 also can include a cutting guide or jig 105D that is used to guide saws or drills used to resect tissue during surgery. Such cutting guides 105D can be formed integrally as part of the effector platform 105 or robotic arm 105A or cutting guides can be separate structures that can be matingly and/or removably attached to the effector platform 105 or robotic arm 105A. The effector platform 105 or robotic arm 105A can be controlled by the CASS 100 to position a cutting guide or jig 105D adjacent to the patient's anatomy in accordance with a pre-operatively or intraoperatively developed surgical plan such that the cutting guide or jig will produce a precise bone cut in accordance with the surgical plan.

The tracking system 115 uses one or more sensors to collect real-time position data that locates the patient's anatomy and surgical instruments. For example, for TKA procedures, the tracking system may provide a location and orientation of the end effector 105B during the procedure. In addition to positional data, data from the tracking system 115 also can be used to infer velocity/acceleration of anatomy/instrumentation, which can be used for tool control. In some embodiments, the tracking system 115 may use a tracker array attached to the end effector 105B to determine the location and orientation of the end effector 105B. The position of the end effector 105B may be inferred based on the position and orientation of the tracking system 115 and a known relationship in three-dimensional space between the tracking system 115 and the end effector 105B. Various types of tracking systems may be used in various embodiments of the present invention including, without limitation, Infrared (IR) tracking systems, electromagnetic (EM) tracking systems, video or image based tracking systems, and ultrasound registration and tracking systems. Using the data provided by the tracking system 115, the surgical computer 150 can detect objects and prevent collision. For example, the surgical computer 150 can prevent the robotic arm 105A and/or the end effector 105B from colliding with soft tissue.

Any suitable tracking system can be used for tracking surgical objects and patient anatomy in the surgical theatre. For example, a combination of IR and visible light cameras can be used in an array. Various illumination sources, such as an IR LED light source, can illuminate the scene allowing three-dimensional imaging to occur. In some embodiments, this can include stereoscopic, tri-scopic, quad-scopic, etc. imaging. In addition to the camera array, which in some embodiments is affixed to a cart, additional cameras can be placed throughout the surgical theatre. For example, handheld tools or headsets worn by operators/surgeons can include imaging capability that communicates images back to a central processor to correlate those images with images captured by the camera array. This can give a more robust image of the environment for modeling using multiple perspectives. Furthermore, some imaging devices may be of suitable resolution or have a suitable perspective on the scene to pick up information stored in quick response (QR) codes or barcodes. This can be helpful in identifying specific objects not manually registered with the system. In some embodiments, the camera may be mounted on the robotic arm 105A.

In some embodiments, specific objects can be manually registered by a surgeon with the system preoperatively or intraoperatively. For example, by interacting with a user interface, a surgeon may identify the starting location for a tool or a bone structure. By tracking fiducial marks associated with that tool or bone structure, or by using other conventional image tracking modalities, a processor may track that tool or bone as it moves through the environment in a three-dimensional model.

In some embodiments, certain markers, such as fiducial marks that identify individuals, important tools, or bones in the theater may include passive or active identifiers that can be picked up by a camera or camera array associated with the tracking system. For example, an IR LED can flash a pattern that conveys a unique identifier to the source of that pattern, providing a dynamic identification mark. Similarly, one- or two-dimensional optical codes (barcode, QR code, etc.) can be affixed to objects in the theater to provide passive identification that can occur based on image analysis. If these codes are placed asymmetrically on an object, they also can be used to determine an orientation of an object by comparing the location of the identifier with the extents of an object in an image. For example, a QR code may be placed in a corner of a tool tray, allowing the orientation and identity of that tray to be tracked. Other tracking modalities are explained throughout. For example, in some embodiments, augmented reality (AR) headsets can be worn by surgeons and other staff to provide additional camera angles and tracking capabilities. In this case, the infrared/time of flight sensor data, which is predominantly used for hand/gesture detection, can build correspondence between the AR headset and the tracking system of the robotic system using sensor fusion techniques. This can be used to calculate a calibration matrix that relates the optical camera coordinate frame to the fixed holographic world frame.

In addition to optical tracking, certain features of objects can be tracked by registering physical properties of the object and associating them with objects that can be tracked, such as fiducial marks fixed to a tool or bone. For example, a surgeon may perform a manual registration process whereby a tracked tool and a tracked bone can be manipulated relative to one another. By impinging the tip of the tool against the surface of the bone, a three-dimensional surface can be mapped for that bone that is associated with a position and orientation relative to the frame of reference of that fiducial mark. By optically tracking the position and orientation (pose) of the fiducial mark associated with that bone, a model of that surface can be tracked with an environment through extrapolation.

The registration process that registers the CASS 100 to the relevant anatomy of the patient also can involve the use of anatomical landmarks, such as landmarks on a bone or cartilage. For example, the CASS 100 can include a 3D model of the relevant bone or joint and the surgeon can intraoperatively collect data regarding the location of bony landmarks on the patient's actual bone using a probe that is connected to the CASS. Bony landmarks can include, for example, the medial malleolus and lateral malleolus, the ends of the proximal femur and distal tibia, and the center of the hip joint. The CASS 100 can compare and register the location data of bony landmarks collected by the surgeon with the probe with the location data of the same landmarks in the 3D model. Alternatively, the CASS 100 can construct a 3D model of the bone or joint without pre-operative image data by using location data of bony landmarks and the bone surface that are collected by the surgeon using a CASS probe or other means. The registration process also can include determining various axes of a joint. For example, for a TKA the surgeon can use the CASS 100 to determine the anatomical and mechanical axes of the femur and tibia. The surgeon and the CASS 100 can identify the center of the hip joint by moving the patient's leg in a spiral direction (i.e., circumduction) so the CASS can determine where the center of the hip joint is located.

A tissue navigation system 120 (not shown in FIG. 1) provides the surgeon with intraoperative, real-time visualization for the patient's bone, cartilage, muscle, nervous, and/or vascular tissues surrounding the surgical area. Examples of systems that may be employed for tissue navigation include fluorescent imaging systems and ultrasound systems.

The display 125 provides graphical user interfaces (GUIs) that display images collected by the tissue navigation system 120 as well other information relevant to the surgery. For example, in one embodiment, the display 125 overlays image information collected from various modalities (e.g., CT, MRI, X-ray, fluorescent, ultrasound, etc.) collected pre-operatively or intra-operatively to give the surgeon various views of the patient's anatomy as well as real-time conditions. The display 125 may include, for example, one or more computer monitors. As an alternative or supplement to the display 125, one or more members of the surgical staff may wear an Augmented Reality (AR) Head Mounted Device (HMD). For example, in FIG. 1 the surgeon 111 is wearing an AR HMD 155 that may, for example, overlay pre-operative image data on the patient or provide surgical planning suggestions. In one embodiment, a tracker array-mounted surgical tool could be detected by both the IR camera and an AR headset (HMD) using sensor fusion techniques without the need for any “intermediate” calibration rigs. This near-depth, time-of-flight sensing camera located in the HMD could be used for hand/gesture detection. The headset's sensor API can be used to expose IR and depth image data and carryout image processing using, for example, C++ with OpenCV. This approach allows the relationship between the CASS and the virtual coordinate frame to be determined and the headset sensor data (i.e., IR in combination with depth images) to isolate the CASS tracker arrays. The image processing system on the HMD can locate the surgical tool in a fixed holographic world frame and the CASS IR camera can locate the surgical tool relative to its camera coordinate frame. This relationship can be used to calculate a calibration matrix that relates the CASS IR camera coordinate frame to the fixed holographic world frame. This means that if a calibration matrix has previously been calculated, the surgical tool no longer needs to be visible to the AR headset. However, a recalculation may be necessary if the CASS camera is accidentally moved in the workflow. Various example uses of the AR HMD 155 in surgical procedures are detailed in the sections that follow.

Surgical computer 150 provides control instructions to various components of the CASS 100, collects data from those components, and provides general processing for various data needed during surgery. In some embodiments, the surgical computer 150 is a general-purpose computer. In other embodiments, the surgical computer 150 may be a parallel computing platform that uses multiple central processing units (CPUs) or graphics processing units (GPU) to perform processing. In some embodiments, the surgical computer 150 is connected to a remote server over one or more computer networks (e.g., the Internet). The remote server can be used, for example, for storage of data or execution of computationally intensive processing tasks.

Various techniques generally known in the art can be used for connecting the surgical computer 150 to the other components of the CASS 100. Moreover, the computers can connect to the surgical computer 150 using a mix of technologies. For example, the end effector 105B may connect to the surgical computer 150 over a wired (i.e., serial) connection. The tracking system 115, tissue navigation system 120, and display 125 can similarly be connected to the surgical computer 150 using wired connections. Alternatively, the tracking system 115, tissue navigation system 120, and display 125 may connect to the surgical computer 150 using wireless technologies such as, without limitation, Wi-Fi, Bluetooth, Near Field Communication (NFC), or ZigBee.

### Robotic Arm

In some embodiments, the CASS 100 includes a robotic arm 105A that serves as an interface to stabilize and hold a variety of instruments used during the surgical procedure. For example, in the context of a hip surgery, these instruments may include, without limitation, retractors, a sagittal or reciprocating saw, the reamer handle, the cup impactor, the broach handle, and the stem inserter. The robotic arm 105A may have multiple degrees of freedom (like a Spider device) and have the ability to be locked in place (e.g., by a press of a button, voice activation, a surgeon removing a hand from the robotic arm, or other method).

In some embodiments, movement of the robotic arm 105A may be effectuated by use of a control panel built into the robotic arm system. For example, a display screen may include one or more input sources, such as physical buttons or a user interface having one or more icons, that direct movement of the robotic arm 105A. The surgeon or other healthcare professional may engage with the one or more input sources to position the robotic arm 105A when performing a surgical procedure.

A tool or an end effector 105B attached or integrated into a robotic arm 105A may include, without limitation, a burring device, a scalpel, a cutting device, a retractor, a joint tensioning device, or the like. In embodiments in which an end effector 105B is used, the end effector may be positioned at the end of the robotic arm 105A such that any motor control operations are performed within the robotic arm system. In embodiments in which a tool is used, the tool may be secured at a distal end of the robotic arm 105A, but motor control operation may reside within the tool itself.

The robotic arm 105A may be motorized internally to both stabilize the robotic arm, thereby preventing it from falling and hitting the patient, surgical table, surgical staff, etc., and to allow the surgeon to move the robotic arm without having to fully support its weight. While the surgeon is moving the robotic arm 105A, the robotic arm may provide some resistance to prevent the robotic arm from moving too fast or having too many degrees of freedom active at once. The position and the lock status of the robotic arm 105A may be tracked, for example, by a controller or the surgical computer 150.

In some embodiments, the robotic arm 105A can be moved by hand (e.g., by the surgeon) or with internal motors into its ideal position and orientation for the task being performed. In some embodiments, the robotic arm 105A may be enabled to operate in a “free” mode that allows the surgeon to position the arm into a desired position without being restricted. While in the free mode, the position and orientation of the robotic arm 105A may still be tracked as described above. In one embodiment, certain degrees of freedom can be selectively released upon input from user (e.g., surgeon) during specified portions of the surgical plan tracked by the surgical computer 150. Designs in which a robotic arm 105A is internally powered through hydraulics or motors or provides resistance to external manual motion through similar means can be described as powered robotic arms, while arms that are manually manipulated without power feedback, but which may be manually or automatically locked in place, may be described as passive robotic arms.

A robotic arm 105A or end effector 105B can include a trigger or other means to control the power of a saw or drill. Engagement of the trigger or other means by the surgeon can cause the robotic arm 105A or end effector 105B to transition from a motorized alignment mode to a mode where the saw or drill is engaged and powered on. Additionally, the CASS 100 can include a foot pedal (not shown) that causes the system to perform certain functions when activated. For example, the surgeon can activate the foot pedal to instruct the CASS 100 to place the robotic arm 105A or end effector 105B in an automatic mode that brings the robotic arm or end effector into the proper position with respect to the patient's anatomy in order to perform the necessary resections. The CASS 100 also can place the robotic arm 105A or end effector 105B in a collaborative mode that allows the surgeon to manually manipulate and position the robotic arm or end effector into a particular location. The collaborative mode can be configured to allow the surgeon to move the robotic arm 105A or end effector 105B medially or laterally, while restricting movement in other directions. As discussed, the robotic arm 105A or end effector 105B can include a cutting device (saw, drill, and burr) or a cutting guide or jig 105D that will guide a cutting device. In other embodiments, movement of the robotic arm 105A or robotically controlled end effector 105B can be controlled entirely by the CASS 100 without any, or with only minimal, assistance or input from a surgeon or other medical professional. In still other embodiments, the movement of the robotic arm 105A or robotically controlled end effector 105B can be controlled remotely by a surgeon or other medical professional using a control mechanism separate from the robotic arm or robotically controlled end effector device, for example using a joystick or interactive monitor or display control device.

The examples below describe uses of the robotic device in the context of a hip surgery; however, it should be understood that the robotic arm may have other applications for surgical procedures involving knees, shoulders, etc. One example of use of a robotic arm in the context of forming an anterior cruciate ligament (ACL) graft tunnel is described in WIPO Publication No. WO 2020/047051, filed Aug. 28, 2019, entitled “Robotic Assisted Ligament Graft Placement and Tensioning,” the entirety of which is incorporated herein by reference.

A robotic arm 105A may be used for holding the retractor. For example, in one embodiment, the robotic arm 105A may be moved into the desired position by the surgeon. At that point, the robotic arm 105A may lock into place. In some embodiments, the robotic arm 105A is provided with data regarding the patient's position, such that if the patient moves, the robotic arm can adjust the retractor position accordingly. In some embodiments, multiple robotic arms may be used, thereby allowing multiple retractors to be held or for more than one activity to be performed simultaneously (e.g., retractor holding & reaming).

The robotic arm 105A may also be used to help stabilize the surgeon's hand while making a femoral neck cut. In this application, control of the robotic arm 105A may impose certain restrictions to prevent soft tissue damage from occurring. For example, in one embodiment, the surgical computer 150 tracks the position of the robotic arm 105A as it operates. If the tracked location approaches an area where tissue damage is predicted, a command may be sent to the robotic arm 105A causing it to stop. Alternatively, where the robotic arm 105A is automatically controlled by the surgical computer 150, the surgical computer may ensure that the robotic arm is not provided with any instructions that cause it to enter areas where soft tissue damage is likely to occur. The surgical computer 150 may impose certain restrictions on the surgeon to prevent the surgeon from reaming too far into the medial wall of the acetabulum or reaming at an incorrect angle or orientation.

In some embodiments, the robotic arm 105A may be used to hold a cup impactor at a desired angle or orientation during cup impaction. When the final position has been achieved, the robotic arm 105A may prevent any further seating to prevent damage to the pelvis.

The surgeon may use the robotic arm 105A to position the broach handle at the desired position and allow the surgeon to impact the broach into the femoral canal at the desired orientation. In some embodiments, once the surgical computer 150 receives feedback that the broach is fully seated, the robotic arm 105A may restrict the handle to prevent further advancement of the broach.

The robotic arm 105A may also be used for resurfacing applications. For example, the robotic arm 105A may stabilize the surgeon while using traditional instrumentation and provide certain restrictions or limitations to allow for proper placement of implant components (e.g., guide wire placement, chamfer cutter, sleeve cutter, plan cutter, etc.). Where only a burr is employed, the robotic arm 105A may stabilize the surgeon's handpiece and may impose restrictions on the handpiece to prevent the surgeon from removing unintended bone in contravention of the surgical plan.

The robotic arm 105A may be a passive arm. As an example, the robotic arm 105A may be a CIRQ robot arm available from Brainlab AG. CIRQ is a registered trademark of Brainlab AG, Olof-Palme-Str. 9 81829, München, FED REP of GERMANY. In one particular embodiment, the robotic arm 105A is an intelligent holding arm as disclosed in U.S. patent application Ser. No. 15/525,585 to Krinninger et al., U.S. patent application Ser. No. 15/561,042 to Nowatschin et al., U.S. patent application Ser. No. 15/561,048 to Nowatschin et al., and U.S. Pat. No. 10,342,636 to Nowatschin et al., the entire contents of each of which is herein incorporated by reference.

### Surgical Procedure Data Generation and Collection

The various services that are provided by medical professionals to treat a clinical condition are collectively referred to as an “episode of care.” For a particular surgical intervention, the episode of care can include three phases: pre-operative, intra-operative, and post-operative. During each phase, data is collected or generated that can be used to analyze the episode of care in order to understand various features of the procedure and identify patterns that may be used, for example, in training models to make decisions with minimal human intervention. The data collected over the episode of care may be stored at the surgical computer 150 or the surgical data server 180 as a complete dataset. Thus, for each episode of care, a dataset exists that comprises all of the data collectively pre-operatively about the patient, all of the data collected or stored by the CASS 100 intra-operatively, and any post-operative data provided by the patient or by a healthcare professional monitoring the patient.

As explained in further detail, the data collected during the episode of care may be used to enhance performance of the surgical procedure or to provide a holistic understanding of the surgical procedure and the patient outcomes. For example, in some embodiments, the data collected over the episode of care may be used to generate a surgical plan. In one embodiment, a high-level, pre-operative plan is refined intra-operatively as data is collected during surgery. In this way, the surgical plan can be viewed as dynamically changing in real-time or near real-time as new data is collected by the components of the CASS 100. In other embodiments, pre-operative images or other input data may be used to develop a robust plan preoperatively that is simply executed during surgery. In this case, the data collected by the CASS 100 during surgery may be used to make recommendations that ensure that the surgeon stays within the pre-operative surgical plan. For example, if the surgeon is unsure how to achieve a certain prescribed cut or implant alignment, the surgical computer 150 can be queried for a recommendation. In still other embodiments, the pre-operative and intra-operative planning approaches can be combined such that a robust pre-operative plan can be dynamically modified, as necessary or desired, during the surgical procedure. In some embodiments, a biomechanics-based model of patient anatomy contributes simulation data to be considered by the CASS 100 in developing preoperative, intraoperative, and post-operative/rehabilitation procedures to optimize implant performance outcomes for the patient.

Aside from changing the surgical procedure itself, the data gathered during the episode of care may be used as an input to other procedures ancillary to the surgery. For example, in some embodiments, implants can be designed using episode of care data. Example data-driven techniques for designing, sizing, and fitting implants are described in U.S. Pat. No. 10,064,686, filed Aug. 15, 2011, and entitled “Systems and Methods for Optimizing Parameters for Orthopaedic Procedures”; U.S. Pat. No. 10,102,309, filed Jul. 20, 2012 and entitled “Systems and Methods for Optimizing Fit of an Implant to Anatomy”; and U.S. Pat. No. 8,078,440, filed Sep. 19, 2008 and entitled “Operatively Tuning Implants for Increased Performance,” the entire contents of each of which are hereby incorporated by reference into this patent application.

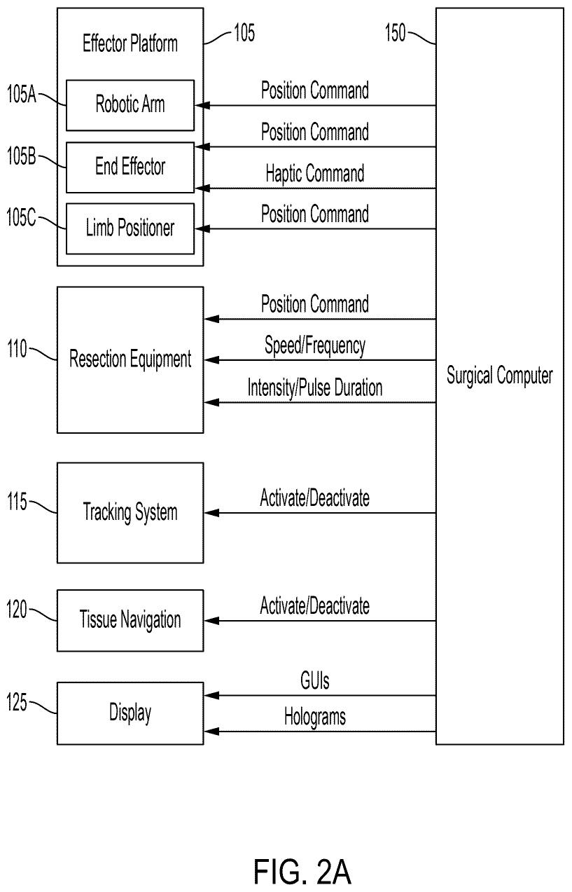

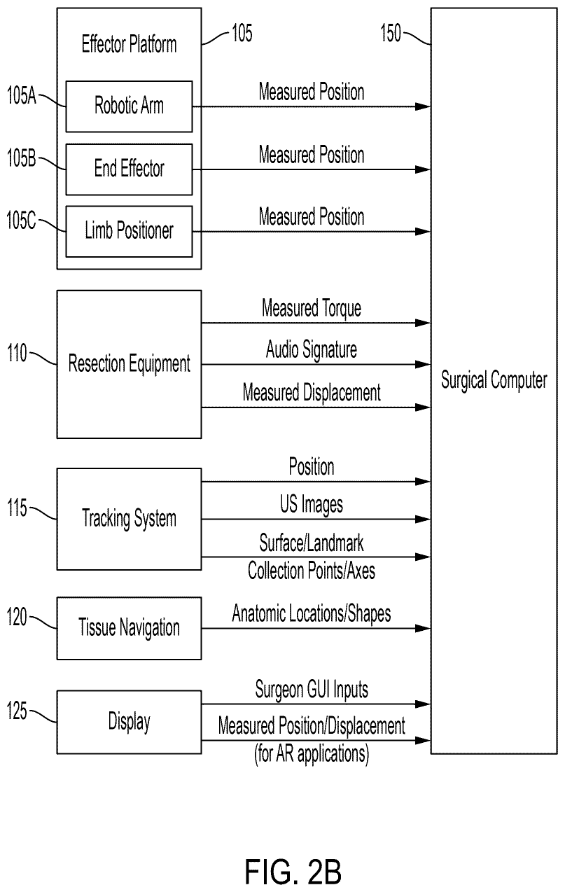

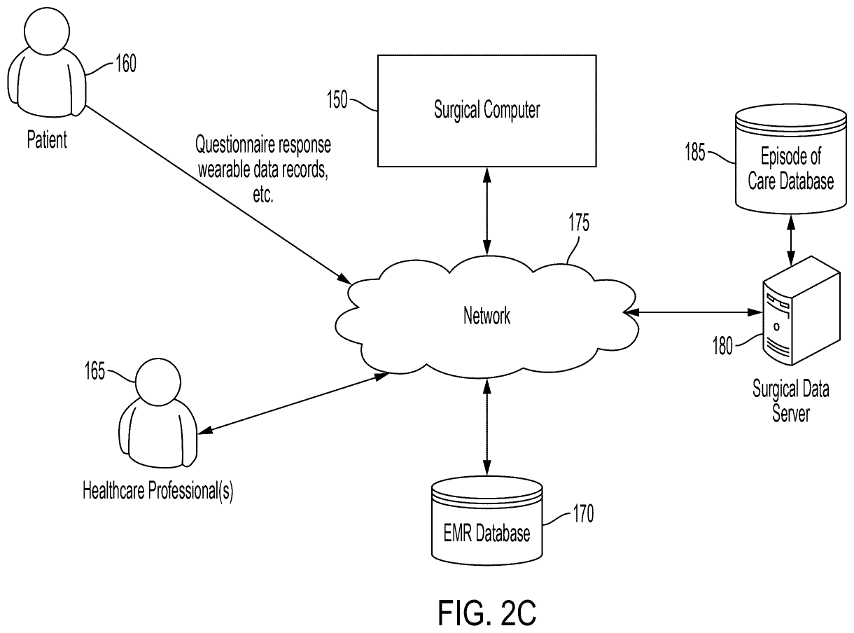

Furthermore, the data can be used for educational, training, or research purposes. For example, using the network-based approach described below in FIG. 2C, other doctors or students can remotely view surgeries in interfaces that allow them to selectively view data as it is collected from the various components of the CASS 100. After the surgical procedure, similar interfaces may be used to “playback” a surgery for training or other educational purposes, or to identify the source of any issues or complications with the procedure.

Data acquired during the pre-operative phase generally includes all information collected or generated prior to the surgery. Thus, for example, information about the patient may be acquired from a patient intake form or electronic medical record (EMR). Examples of patient information that may be collected include, without limitation, patient demographics, diagnoses, medical histories, progress notes, vital signs, medical history information, allergies, and lab results. The pre-operative data may also include images related to the anatomical area of interest. These images may be captured, for example, using Magnetic Resonance Imaging (MRI), Computed Tomography (CT), X-ray, ultrasound, or any other modality known in the art. The pre-operative data may also comprise quality of life data captured from the patient. For example, in one embodiment, pre-surgery patients use a mobile application (“app”) to answer questionnaires regarding their current quality of life. In some embodiments, preoperative data used by the CASS 100 includes demographic, anthropometric, cultural, or other specific traits about a patient that can coincide with activity levels and specific patient activities to customize the surgical plan to the patient. For example, certain cultures or demographics may be more likely to use a toilet that requires squatting on a daily basis.

FIGS. 2A and 2B provide examples of data that may be acquired during the intra-operative phase of an episode of care. These examples are based on the various components of the CASS 100 described above with reference to FIG. 1; however, it should be understood that other types of data may be used based on the types of equipment used during surgery and their use.

FIG. 2A shows examples of some of the control instructions that the surgical computer 150 provides to other components of the CASS 100, according to some embodiments. Note that the example of FIG. 2A assumes that the components of the effector platform 105 are each controlled directly by the surgical computer 150. In embodiments where a component is manually controlled by the Surgeon 111, instructions may be provided on the Display 125 or AR HMD 155 instructing the Surgeon 111 how to move the component.

The various components included in the effector platform 105 are controlled by the surgical computer 150 providing position commands that instruct the component where to move within a coordinate system. In some embodiments, the surgical computer 150 provides the effector platform 105 with instructions defining how to react when a component of the effector platform 105 deviates from a surgical plan. These commands are referenced in FIG. 2A as “haptic” commands. For example, the end effector 105B may provide a force to resist movement outside of an area where resection is planned. Other commands that may be used by the effector platform 105 include vibration and audio cues.

In some embodiments, the end effectors 105B of the robotic arm 105A are operatively coupled with cutting guide 105D. In response to an anatomical model of the surgical scene, the robotic arm 105A can move the end effectors 105B and the cutting guide 105D into position to match the location of the femoral or tibial cut to be performed in accordance with the surgical plan. This can reduce the likelihood of error, allowing the vision system and a processor utilizing that vision system to implement the surgical plan to place a cutting guide 105D at the precise location and orientation relative to the tibia or femur to align a cutting slot of the cutting guide with the cut to be performed according to the surgical plan. Then, a surgeon can use any suitable tool, such as an oscillating or rotating saw or drill to perform the cut (or drill a hole) with perfect placement and orientation because the tool is mechanically limited by the features of the cutting guide 105D. In some embodiments, the cutting guide 105D may include one or more pin holes that are used by a surgeon to drill and screw or pin the cutting guide into place before performing a resection of the patient tissue using the cutting guide. This can free the robotic arm 105A or ensure that the cutting guide 105D is fully affixed without moving relative to the bone to be resected. For example, this procedure can be used to make the first distal cut of the femur during a total knee arthroplasty. In some embodiments, where the arthroplasty is a hip arthroplasty, cutting guide 105D can be fixed to the femoral head or the acetabulum for the respective hip arthroplasty resection. It should be understood that any arthroplasty that utilizes precise cuts can use the robotic arm 105A and/or cutting guide 105D in this manner.

The resection equipment 110 is provided with a variety of commands to perform bone or tissue operations. As with the effector platform 105, position information may be provided to the resection equipment 110 to specify where it should be located when performing resection. Other commands provided to the resection equipment 110 may be dependent on the type of resection equipment. For example, for a mechanical or ultrasonic resection tool, the commands may specify the speed and frequency of the tool. For Radiofrequency Ablation (RFA) and other laser ablation tools, the commands may specify intensity and pulse duration.

Some components of the CASS 100 do not need to be directly controlled by the surgical computer 150; rather, the surgical computer 150 only needs to activate the component, which then executes software locally specifying the manner in which to collect data and provide it to the surgical computer 150. In the example of FIG. 2A, there are two components that are operated in this manner: the tracking system 115 and the tissue navigation system 120.

The surgical computer 150 provides the display 125 with any visualization that is needed by the surgeon 111 during surgery. For monitors, the surgical computer 150 may provide instructions for displaying images, GUIs, etc. using techniques known in the art. The display 125 can include various portions of the workflow of a surgical plan. During the registration process, for example, the display 125 can show a preoperatively constructed 3D bone model and depict the locations of the probe as the surgeon uses the probe to collect locations of anatomical landmarks on the patient. The display 125 can include information about the surgical target area. For example, in connection with a TKA, the display 125 can depict the mechanical and anatomical axes of the femur and tibia. The display 125 can depict varus and valgus angles for the knee joint based on a surgical plan, and the CASS 100 can depict how such angles will be affected if contemplated revisions to the surgical plan are made. Accordingly, the display 125 is an interactive interface that can dynamically update and display how changes to the surgical plan would impact the procedure and the final position and orientation of implants installed on bone.

As the workflow progresses to preparation of bone cuts or resections, the display 125 can depict the planned or recommended bone cuts before any cuts are performed. The surgeon 111 can manipulate the image display to provide different anatomical perspectives of the target area and can have the option to alter or revise the planned bone cuts based on intraoperative evaluation of the patient. The display 125 can depict how the chosen implants would be installed on the bone if the planned bone cuts are performed. If the surgeon 111 choses to change the previously planned bone cuts, the display 125 can depict how the revised bone cuts would change the position and orientation of the implant when installed on the bone.

The display 125 can provide the surgeon 111 with a variety of data and information about the patient, the planned surgical intervention, and the implants. Various patient-specific information can be displayed, including real-time data concerning the patient's health such as heart rate, blood pressure, etc. The display 125 also can include information about the anatomy of the surgical target region including the location of landmarks, the current state of the anatomy (e.g., whether any resections have been made, the depth and angles of planned and executed bone cuts), and future states of the anatomy as the surgical plan progresses. The display 125 also can provide or depict additional information about the surgical target region. For a TKA, the display 125 can provide information about the gaps (e.g., gap balancing) between the femur and tibia and how such gaps will change if the planned surgical plan is carried out. For a TKA, the display 125 can provide additional relevant information about the knee joint such as data about the joint's tension (e.g., ligament laxity) and information concerning rotation and alignment of the joint. The display 125 can depict how the planned implants' locations and positions will affect the patient as the knee joint is flexed. The display 125 can depict how the use of different implants or the use of different sizes of the same implant will affect the surgical plan and preview how such implants will be positioned on the bone. The CASS 100 can provide such information for each of the planned bone resections in a TKA or THA. In a TKA, the CASS 100 can provide robotic control for one or more of the planned bone resections. For example, the CASS 100 can provide robotic control only for the initial distal femur cut, and the surgeon 111 can manually perform other resections (anterior, posterior and chamfer cuts) using conventional means, such as a 4-in-1 cutting guide or jig 105D.

The display 125 can employ different colors to inform the surgeon of the status of the surgical plan. For example, un-resected bone can be displayed in a first color, resected bone can be displayed in a second color, and planned resections can be displayed in a third color. Implants can be superimposed onto the bone in the display 125, and implant colors can change or correspond to different types or sizes of implants.

The information and options depicted on the display 125 can vary depending on the type of surgical procedure being performed. Further, the surgeon 111 can request or select a particular surgical workflow display that matches or is consistent with his or her surgical plan preferences. For example, for a surgeon 111 who typically performs the tibial cuts before the femoral cuts in a TKA, the display 125 and associated workflow can be adapted to take this preference into account. The surgeon 111 also can preselect that certain steps be included or deleted from the standard surgical workflow display. For example, if a surgeon 111 uses resection measurements to finalize an implant plan but does not analyze ligament gap balancing when finalizing the implant plan, the surgical workflow display can be organized into modules, and the surgeon can select which modules to display and the order in which the modules are provided based on the surgeon's preferences or the circumstances of a particular surgery. Modules directed to ligament and gap balancing, for example, can include pre- and post-resection ligament/gap balancing, and the surgeon 111 can select which modules to include in their default surgical plan workflow depending on whether they perform such ligament and gap balancing before or after (or both) bone resections are performed.

For more specialized display equipment, such as AR HMDs, the surgical computer 150 may provide images, text, etc. using the data format supported by the equipment. For example, if the Display 125 is a holography device such as the Microsoft HoloLens™ or Magic Leap One™, the surgical computer 150 may use the HoloLens Application Program Interface (API) to send commands specifying the position and content of holograms displayed in the field of view of the surgeon 111.

In some embodiments, one or more surgical planning models may be incorporated into the CASS 100 and used in the development of the surgical plans provided to the surgeon 111. The term “surgical planning model” refers to software that simulates the biomechanics performance of anatomy under various scenarios to determine the optimal way to perform cutting and other surgical activities. For example, for knee replacement surgeries, the surgical planning model can measure parameters for functional activities, such as deep knee bends, gait, etc., and select cut locations on the knee to optimize implant placement. One example of a surgical planning model is the LIFEMOD™ simulation software from SMITH AND NEPHEW, INC. In some embodiments, the surgical computer 150 includes computing architecture that allows full execution of the surgical planning model during surgery (e.g., a GPU-based parallel processing environment). In other embodiments, the surgical computer 150 may be connected over a network to a remote computer that allows such execution, such as a Surgical Data Server 180 (see FIG. 2C). As an alternative to full execution of the surgical planning model, in some embodiments, a set of transfer functions are derived that simplify the mathematical operations captured by the model into one or more predictor equations. Then, rather than execute the full simulation during surgery, the predictor equations are used. Further details on the use of transfer functions are described in WIPO Publication No. 2020/037308, filed Aug. 19, 2019, entitled “Patient Specific Surgical Method and System,” the entirety of which is incorporated herein by reference.

FIG. 2B shows examples of some of the types of data that can be provided to the surgical computer 150 from the various components of the CASS 100. In some embodiments, the components may stream data to the surgical computer 150 in real-time or near real-time during surgery. In other embodiments, the components may queue data and send it to the surgical computer 150 at set intervals (e.g., every second). Data may be communicated using any format known in the art. Thus, in some embodiments, the components all transmit data to the surgical computer 150 in a common format. In other embodiments, each component may use a different data format, and the surgical computer 150 is configured with one or more software applications that enable translation of the data.

In general, the surgical computer 150 may serve as the central point where CASS data is collected. The exact content of the data will vary depending on the source. For example, each component of the effector platform 105 provides a measured position to the surgical computer 150. Thus, by comparing the measured position to a position originally specified by the surgical computer 150 (see FIG. 2B), the surgical computer 150 can identify deviations that take place during surgery.

The resection equipment 110 can send various types of data to the surgical computer 150 depending on the type of equipment used. Example data types that may be sent include the measured torque, audio signatures, and measured displacement values. Similarly, the tracking technology 115 can provide different types of data depending on the tracking methodology employed. Example tracking data types include position values for tracked items (e.g., anatomy, tools, etc.), ultrasound images, and surface or landmark collection points or axes. The Tissue Navigation System 120 provides the surgical computer 150 with anatomic locations, shapes, etc. as the system operates.

Although the display 125 generally is used for outputting data for presentation to the user, it may also provide data to the surgical computer 150. For example, for embodiments where a monitor is used as part of the display 125, the surgeon 111 may interact with a GUI to provide inputs which are sent to the surgical computer 150 for further processing. For AR applications, the measured position and displacement of the HMD may be sent to the surgical computer 150 so that it can update the presented view as needed.

During the post-operative phase of the episode of care, various types of data can be collected to quantify the overall improvement or deterioration in the patient's condition as a result of the surgery. The data can take the form of, for example, self-reported information reported by patients via questionnaires. For example, in the context of a knee replacement surgery, functional status can be measured with an Oxford Knee Score questionnaire, and the post-operative quality of life can be measured with a EQ5D-5L questionnaire. Other examples in the context of a hip replacement surgery may include the Oxford Hip Score, Harris Hip Score, and WOMAC (Western Ontario and McMaster Universities Osteoarthritis index). Such questionnaires can be administered, for example, by a healthcare professional directly in a clinical setting or using a mobile app that allows the patient to respond to questions directly. In some embodiments, the patient may be outfitted with one or more wearable devices that collect data relevant to the surgery. For example, following a knee surgery, the patient may be outfitted with a knee brace that includes sensors that monitor knee positioning, flexibility, etc. This information can be collected and transferred to the patient's mobile device for review by the surgeon to evaluate the outcome of the surgery and address any issues. In some embodiments, one or more cameras can capture and record the motion of a patient's body segments during specified activities postoperatively. This motion capture can be compared to a biomechanics model to better understand the functionality of the patient's joints and better predict progress in recovery and identify any possible revisions that may be needed.

The post-operative stage of the episode of care can continue over the entire life of a patient. For example, in some embodiments, the surgical computer 150 or other components comprising the CASS 100 can continue to receive and collect data relevant to a surgical procedure after the procedure has been performed. This data may include, for example, images, answers to questions, “normal” patient data (e.g., blood type, blood pressure, conditions, medications, etc.), biometric data (e.g., gait, etc.), and objective and subjective data about specific issues (e.g., knee or hip joint pain). This data may be explicitly provided to the surgical computer 150 or other CASS component by the patient or the patient's physician(s). Alternatively, or additionally, the surgical computer 150 or other CASS component can monitor the patient's EMR and retrieve relevant information as it becomes available. This longitudinal view of the patient's recovery allows the surgical computer 150 or other CASS component to provide a more objective analysis of the patient's outcome to measure and track success or lack of success for a given procedure. For example, a condition experienced by a patient long after the surgical procedure can be linked back to the surgery through a regression analysis of various data items collected during the episode of care. This analysis can be further enhanced by performing the analysis on groups of patients that had similar procedures and/or have similar anatomies.

In some embodiments, data is collected at a central location to provide for easier analysis and use. Data can be manually collected from various CASS components in some instances. For example, a portable storage device (e.g., USB stick) can be attached to the surgical computer 150 into order to retrieve data collected during surgery. The data can then be transferred, for example, via a desktop computer to the centralized storage. Alternatively, in some embodiments, the surgical computer 150 is connected directly to the centralized storage via a network 175 as shown in FIG. 2C.

FIG. 2C illustrates a “cloud-based” implementation in which the surgical computer 150 is connected to a surgical data server 180 via a network 175. This Network 175 may be, for example, a private intranet or the Internet. In addition to the data from the surgical computer 150, other sources can transfer relevant data to the Surgical Data Server 180. The example of FIG. 2C shows three additional data sources: the patient 160, healthcare professional(s) 165, and an EMR Database 170. Thus, the patient 160 can send pre-operative and post-operative data to the surgical data server 180, for example, using a mobile app. The Healthcare Professional(s) 165 includes the surgeon and his or her staff as well as any other professionals working with Patient 160 (e.g., a personal physician, a rehabilitation specialist, etc.). It should also be noted that the EMR Database 170 may be used for both pre-operative and post-operative data. For example, assuming that the Patient 160 has given adequate permissions, the Surgical Data Server 180 may collect the EMR of the Patient pre-surgery. Then, the Surgical Data Server 180 may continue to monitor the EMR for any updates post-surgery.

At the Surgical Data Server 180, an Episode of Care Database 185 is used to store the various data collected over a patient's episode of care. The Episode of Care Database 185 may be implemented using any technique known in the art. For example, in some embodiments, a SQL-based database may be used where all of the various data items are structured in a manner that allows them to be readily incorporated in two SQL's collection of rows and columns. However, in other embodiments a No-SQL database may be employed to allow for unstructured data, while providing the ability to rapidly process and respond to queries. As is understood in the art, the term “No-SQL” is used to define a class of data stores that are non-relational in their design. Various types of No-SQL databases may generally be grouped according to their underlying data model. These groupings may include databases that use column-based data models (e.g., Cassandra), document-based data models (e.g., MongoDB), key-value based data models (e.g., Redis), and/or graph-based data models (e.g., Allego). Any type of No-SQL database may be used to implement the various embodiments described herein and, in some embodiments, the different types of databases may support the Episode of Care Database 185.

Data can be transferred between the various data sources and the Surgical Data Server 180 using any data format and transfer technique known in the art. It should be noted that the architecture shown in FIG. 2C allows transmission from the data source to the Surgical Data Server 180, as well as retrieval of data from the Surgical Data Server 180 by the data sources. For example, as explained in detail below, in some embodiments, the surgical computer 150 may use data from past surgeries, machine learning models, etc. to help guide the surgical procedure.

In some embodiments, the surgical computer 150 or the Surgical Data Server 180 may execute a de-identification process to ensure that data stored in the Episode of Care Database 185 meets Health Insurance Portability and Accountability Act (HIPAA) standards or other requirements mandated by law. HIPAA provides a list of certain identifiers that must be removed from data during de-identification. The aforementioned de-identification process can scan for these identifiers in data that is transferred to the Episode of Care Database 185 for storage. For example, in one embodiment, the surgical computer 150 executes the de-identification process just prior to initiating transfer of a particular data item or set of data items to the Surgical Data Server 180. In some embodiments, a unique identifier is assigned to data from a particular episode of care to allow for re-identification of the data if necessary.

Although FIGS. 2A-C discuss data collection in the context of a single episode of care, it should be understood that the general concept can be extended to data collection from multiple episodes of care. For example, surgical data may be collected over an entire episode of care each time a surgery is performed with the CASS 100 and stored at the surgical computer 150 or at the Surgical Data Server 180. As explained in further detail below, a robust database of episode of care data allows the generation of optimized values, measurements, distances, or other parameters and other recommendations related to the surgical procedure. In some embodiments, the various datasets are indexed in the database or other storage medium in a manner that allows for rapid retrieval of relevant information during the surgical procedure. For example, in one embodiment, a patient-centric set of indices may be used so that data pertaining to a particular patient or a set of patients similar to a particular patient can be readily extracted. This concept can be similarly applied to surgeons, implant characteristics, CASS component versions, etc.

Further details of the management of episode of care data are described in U.S. patent application Ser. No. 16/847,183, filed Apr. 13, 2020, published as U.S. Publication No. 2020/0243199, and entitled “METHODS AND SYSTEMS FOR PROVIDING AN EPISODE OF CARE,” the entirety of which is incorporated herein by reference.

According to an aspect of the present disclosure, certain surgical contexts, mechanical resection tools such as oscillating saws and rotary burrs can generate excessive heat and debris. External water cooling is often used to mitigate frictional heating; however, sclerotic bone lesions can exacerbate thermal load during mechanical osteotomy, increasing thermal necrosis. Mechanical resection can also produce significant bone debris (sequestra) that delays bone regeneration and presents a biohazard if aerosolized, increasing risks to staff and patients.

Laser resection offers several benefits relative to mechanical resection, including high precision, clean and contact-free cuts that reduce mechanical loading, flexible cut geometries, the absence of metal tool debris, lower surface roughness, and reduced smear layer at osteotomy edges. Nevertheless, traditional focused-beam medical laser systems have been limited by relatively low processing speeds, difficulty achieving planar cuts, and depth-dependent efficiency losses when the laser-tissue standoff is large and debris is not effectively controlled in deep craters.

The present disclosure addresses these issues by including optimized beam shaping (e.g., a top-hat intensity profile), localized mist cooling and suction to maintain a clear optical path in deep craters, and a multiplexed fiber delivery system configured to operate the end effector at a fixed, near-surface standoff. These features support deeper, faster, and safer bone resection while maintaining tissue viability.

### Orthopedic Oscillating Saws

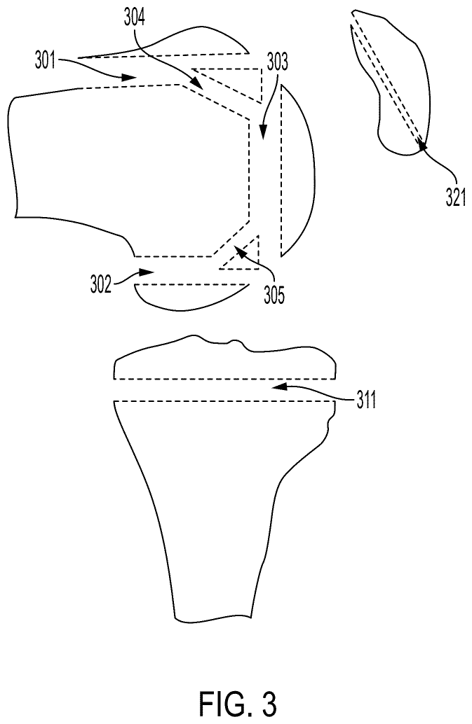

FIG. 3 illustrates example bone cuts in a bone cutting procedure, such as a typical total knee arthroplasty (TKA) procedure in accordance with an embodiment. In this example, the bone cuts can include one or more of the distal femur 303, anterior femur 301, posterior femur 302, anterior chamfer 304, posterior chamfer 305, tibia 311, and patella 321 cuts. Planar cuts can exceed 50 mm in depth for the tibia 311, 30 mm for the five box cuts of the femur 301-305 and 20 mm for the patella 321 for a total knee replacement surgery. The anterior femoral cut 301 can set the femoral component rotation, which can have a direct effect upon patellar tracking and gap balancing. The posterior condyle 302 and tibial 311 cuts can affect the flexion gap. Other cutting depths and/or parameters may also be possible according to various aspects of the present disclosure.

Orthopedic oscillating saws are widely used for plane processing during joint replacement procedures with bone removal rates of approximately 5 mm³/s. However, a sagittal saw can cause irregular crack propagation and fractured bone chips, which in turn affects the tissue removal process and postoperative recovery.

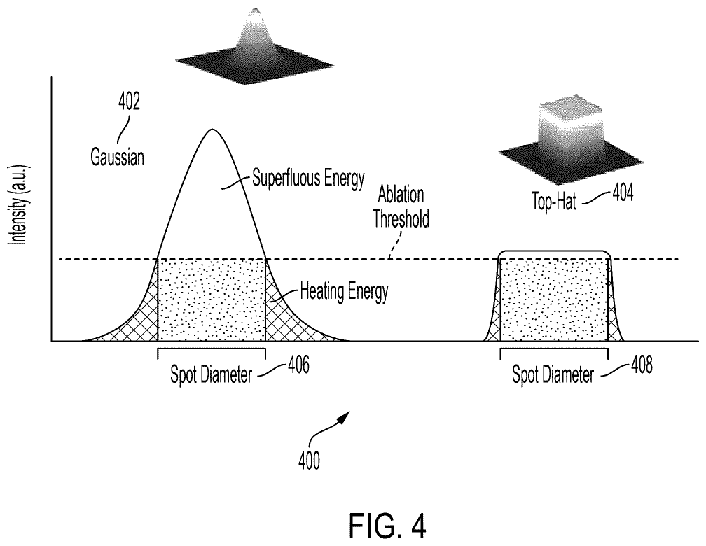

Referring to FIG. 4, a graph 400 is depicted comparing the intensity distribution profiles of a Gaussian beam 402 and a non-Gaussian, top-hat beam 404 as used in laser bone ablation. Prior to the present disclosure, conventional laser systems typically relied on Gaussian-like output intensity distributions, which resulted in a high central intensity and a rapid fall-off toward the edges of the beam spot. These side lobes usually do not contribute to the ablation process, and instead cause a temperature increase in the surrounding tissues, which can increase the risk of thermal necrosis. This generally results in a conical-shaped ablation profile in bone, caused by spatial filtering of the cut walls as the depth increases. This configuration often led to challenges in delivering the beam through optical fibers, as the central fluence could exceed the damage threshold of the fiber even when the average power remained below the threshold. As a result, fibers could be damaged at energy levels (in excess of 500 mJ) that would otherwise be considered safe, limiting the efficiency and/or reliability of laser-based bone ablation systems.

The present disclosure overcomes these limitations by including a beam shaping strategy that utilizes specially designed optics or diffractive elements to generate a top-hat beam profile. This approach enables more uniform energy delivery across the spot diameter 408, reducing superfluous energy above the ablation threshold and minimizing heating energy below the threshold, thereby improving ablation efficiency and fiber durability. For example, a uniform intensity or tophat intensity distribution delivers ablation-capable fluence across its entire cross-section. Especially if the edges of the beam are steep, the resulting ablation profile can be expected to have a flat bottom, following the beam profile. According to an implementation, the process begins with an average fluence just above the ablation threshold, focuses within the bone, and continues until the average fluence drops below the threshold. Although such a beam might be expected to produce a roughly symmetrical depth profile, a maximum depth can be achieved when the focal plane is positioned ˜10 mm below the bone surface. When the bone is positioned far before the focal plane, the enlarged spot reduced fluence and efficiency with increasing depth, preventing maximum penetration.

The graph 400 includes an example representation of the spot diameter for both the Gaussian 406 and top-hat beams 408. The Gaussian beam profile is characterized by including an intensity profile 402 detailing a pronounced peak at the center, where the intensity is highest, and a decrease in intensity toward the periphery. This results in a central region where the fluence is above the ablation threshold, indicated as superfluous energy, and outer regions where the energy is insufficient for ablation, leading to wasted heating energy. The top-hat beam profile, in contrast, is depicted as having an intensity profile 404 including a relatively flat intensity distribution across the spot diameter 408, with reduced deviation from the ablation threshold. In this configuration, the energy delivered to the target is more consistent as compared to the Gaussian beam profile, with the majority of the beam area operating at or just above the ablation threshold, and reduced superfluous energy at the center.

According to an aspect of the disclosure, the ablation threshold is indicated on the graph 400 as a reference level for both beam profiles. For the Gaussian beam, the area above the threshold represents energy that does not contribute to efficient ablation and may cause unwanted heating and/or damage to the fiber. The area below the threshold, such as at the edges of the spot, represents energy that is insufficient for ablation and is therefore wasted. For the top-hat beam, the energy distribution closely matches the ablation threshold across the spot diameter 408, resulting in more effective use of the delivered energy and reduced risk of fiber damage.

According to the embodiments of the present disclosures the use of beam shaping optics or diffractive elements to achieve the top-hat intensity profile, as represented in the graph 400, may function to redistribute the laser energy such that the output beam maintains a uniform intensity across the spot, rather than concentrating energy at the center. Here, a uniform intensity may mean that the intensity various is within 1%, 2%, 5%, 10%, or other percentage. By minimizing superfluous energy and maximizing the proportion of the beam operating at the ablation threshold, the present disclosure enables deeper and faster bone ablation, improved fiber longevity, and more predictable surgical outcomes. The interaction between the beam shaping elements and the optical fiber delivery system ensures that the laser energy is transmitted efficiently and safely to the target tissue, supporting the overall objectives of the present disclosure.

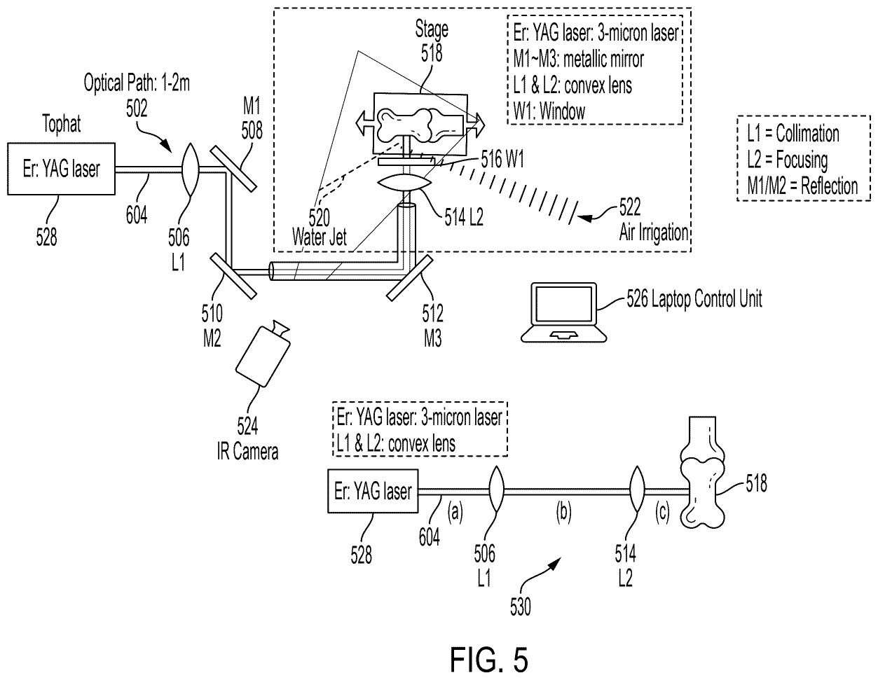

According to another aspect of the present disclosure, specially designed optics or diffractive elements may be introduced (as shown in FIG. 5 and elsewhere) to achieve the transformation from a Gaussian beam 402 to a top-hat beam 404. These elements function to redistribute the laser energy such that the output beam maintains a uniform intensity across the spot, rather than concentrating energy at the center. By optimizing the spatial energy distribution in this manner, the present disclosure enables more efficient use of the delivered energy and supports greater ablation depth. The interaction between the beam shaping elements and the laser source ensures that the energy is transmitted efficiently to the target tissue, supporting the objectives of precise, rapid, and controlled bone ablation.

Referring to FIG. 5, a laser delivery and beam shaping system 500 is provided for delivering a single laser spot with beam shaping and refined optics to achieve a top-hat beam profile for bone ablation. The present disclosure includes an arrangement 500 in which an Er:YAG laser 528, operating at a wavelength of approximately 3 microns, emits a laser beam 604 that is directed along an optical path 502. Other types and/or wavelengths for the laser may also be used according to various aspects of the present disclosure. The optical path 502 is configured to extend over a distance of approximately 1 to 2 meters and includes a series of optical components for collimation, reflection, and focusing of the laser beam 604.

In an aspect, free-space ablation testing with optimized optics, a top-hat beam intensity profile achieved an ablation depth of approximately 42 mm at comparable pulse energy where a Gaussian beam was limited to approximately 27.5 mm. Surface removal rates for top-hat beams were measured at approximately 2-2.5 mm³/s in dense cortical bone, versus approximately 1-1.5 mm³/s reported for prior systems at similar operating conditions. The removal may be performed across air, air-plus-water, and mist-cooled states, and the rates are attributed to efficient energy use above the ablation threshold with reduced superfluous heating outside the ablation zone.

Additional strategies that further increase ablation depth and rate include widening the near cortex to enhance penetration of mist and air into the crater, optimizing water duty cycles, and amplifying delivered energy via multiplexed fiber output. In certain tests, top-hat beam profile deviation remained within approximately ±5%, supporting consistent energy delivery with depth.

According to an aspect, the laser beam 604 is first directed through a collimation lens L1 506, which is a convex lens configured to collimate the output of the Er:YAG laser. The collimated beam 604 is then sequentially reflected by metallic mirrors M1 508, M2 510, and M3 512, which are arranged to guide the beam 604 along the defined optical path 502. After reflection, the beam 604 passes through a focusing lens L2 514, which is also a convex lens, and is configured to focus the collimated beam 604 to a desired spot size suitable for ablation. The focused beam 604 may then pass through a window (W1) 516, which serves as an optical interface and environmental barrier, before reaching the bone 518. In some implementations, the window 516 may be protective and/or transparent.

The present disclosure may further include a waterjet 520 positioned to deliver irrigation fluid directly to the bone 518 at the ablation site. In addition, air irrigation 522 is provided to assist in cooling the ablation site and removing debris via evacuating suction, thereby maintaining a clear optical path and reducing the risk of thermal damage to surrounding tissue. An infrared (IR) camera 524 is positioned to monitor the laser beam 604 in real-time or substantially real-time, enabling precise alignment and quality assurance of the beam 604 profile during operation. A laptop or control unit 526 may be configured to orchestrate the operation of the system 500, including control of the laser emission, optical alignment, irrigation delivery, and monitoring functions. The arrangement of these elements ensures that laser energy is delivered efficiently and safely to the bone 518, supporting rapid, deep, and controlled bone removal as described in the present disclosure.

Referring further to FIG. 5, the present disclosure also includes an alternate optical pathway 530 in which the laser beam 604 from the Er:YAG laser 528 is directed through only the collimation lens L1 506 and the focusing lens L2 514 before reaching the bone 518. In this configuration, the beam 604 bypasses the sequence of metallic mirrors and window, providing a more direct optical path from the laser source to the target tissue. This arrangement can be selected based on procedural requirements or system constraints, and is contemplated as part of the present disclosure.

The present disclosure includes the use of a wide range of laser sources and configurations suitable for bone ablation and related surgical procedures. As a non-limiting example, the present disclosure includes a laser system 528, such as the 3 micron Laser Technology Inc. 30 W 3 μm laser, which is configured with integrated beam shaping. This system is capable of delivering an average power of 30 W and a peak power of 1.6 kW at a wavelength of 2.94 μm. The laser output is 0.8 J at a pulse duration of 0.5 milliseconds and a repetition rate of 36 Hz, with the pulse duration being adjustable from 250 to 500 microseconds and the repetition rate adjustable from 36 to 72 Hz. The physical dimensions of this laser system are approximately 12 inches in length, 1.75 inches in width, and 2.25 inches in height, excluding the power connector, and the weight is less than 1 kilogram without the power connector.

The present disclosure also includes the use of other suitable laser sources 528, such as the Pantec Biosolutions AG DPM-80 laser, which operates at an ER:YAG wavelength and includes integrated beam shaping. The DPM-80 has dimensions of 90×78×59 millimeters, excluding the power connector, and a weight of less than 1 kilogram without the power connector. The pulse duration is adjustable from 40 to 1000 microseconds, and the system is capable of delivering a pulsed energy of up to 3.5 joules (3.4 joules per pulse). After transmission through the fiber, the output is 400 millijoules, and when two lasers are used in combination, the output is 800 millijoules, with a maximum of 2.5 joules per pulse.

These examples are provided for illustrative purposes only, and the present disclosure is not limited to any particular laser source, wavelength, power level, pulse duration, or physical configuration. Any laser system capable of delivering controlled energy to bone or hard tissue, with or without integrated beam shaping, is contemplated within the scope of the present disclosure.

According to an embodiment of the present disclosure, operating windows for deep ablation include microsecond pulse durations (e.g., 100-500 μs) below the thermal relaxation time for bone, pulse repetition rates selected to limit cumulative heating and to interface with automated mist and suction duty cycles, and wavelengths near 2.94 μm to leverage absorption peaks of water and hydroxyapatite. Alternative ranges, including operation at lower repetition rates (e.g., <20 Hz for certain multiplexed fiber configurations) help avoid premature fiber degradation. Where power scaling is used, fiber-coupled outputs may be tuned by drive current to achieve desired per-pulse energy (e.g., representative fiber-coupling tests demonstrated incremental pulse energy increases with current until fiber tip damage thresholds were reached), with in-line power metering used for verification.

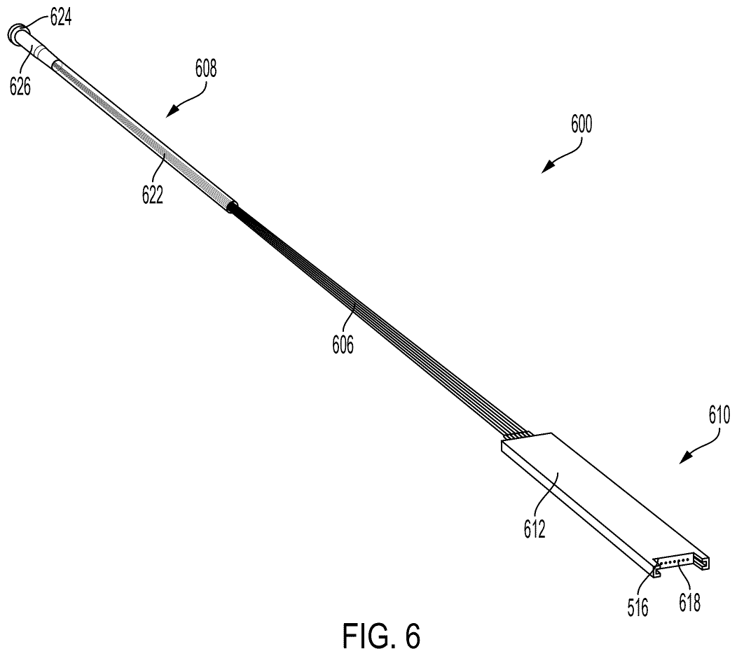

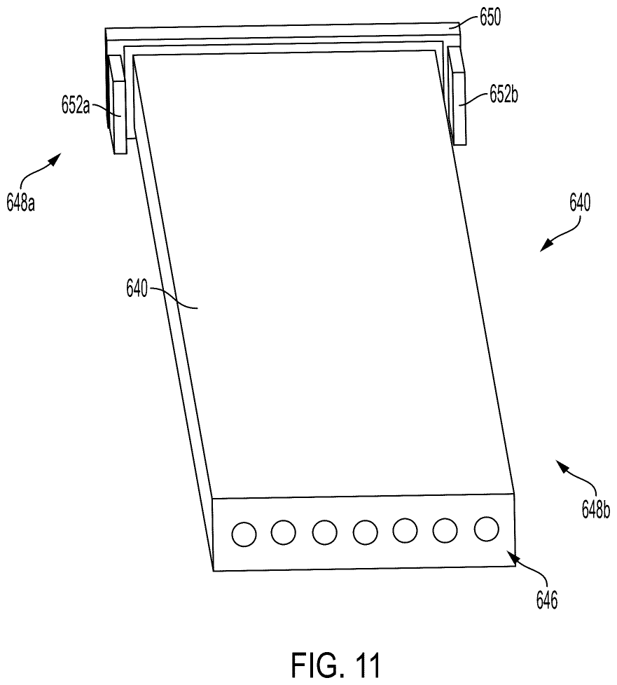

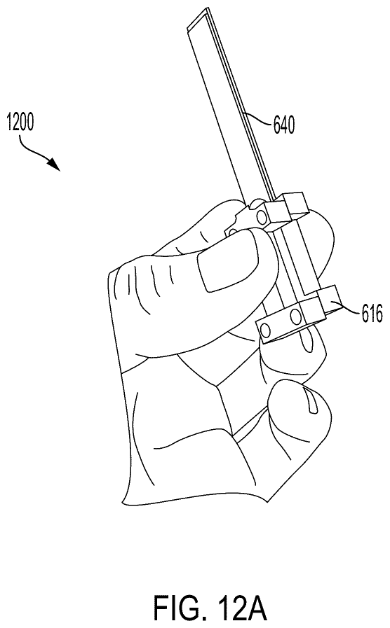

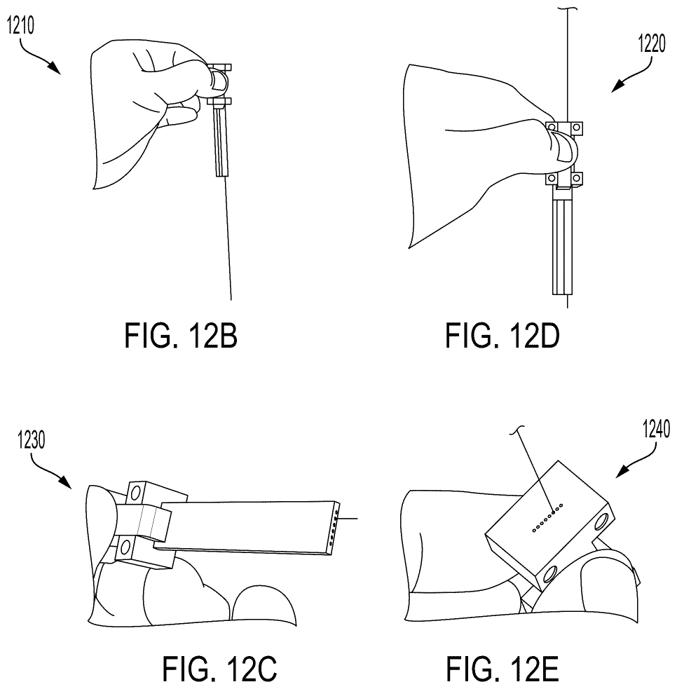

Referring to FIG. 6, an apparatus 600 is provided for resecting hard biological tissue 620, such as bone. The present disclosure includes a laser source 528 configured to emit a laser beam 604. The laser beam 604 is optically coupled to a plurality of optical fibers 606, each optical fiber 606 having a fused proximal end 608 optically coupled to the laser source 528 and a distal end 610 configured to emit the laser beam 604 toward the tissue 620. According to an implementation, the plurality of optical fibers may include a bundle diameter of 1-2 mm. The plurality of optical fibers 606 are supported by a support structure 616 (shown in FIGS. 11-12E), which may be integrated with the cartridge 640 and/or the sterile outer-housing (spacing element 612), and is configured to maintain the distal ends 610 of the optical fibers 606 in a predetermined spatial arrangement. More specifically, the cartridge 640 may be inserted into the spacing element 612, and the support structure 616 is responsible for the precise arrangement of the fiber tips at the ablation interface. In an example, a beam shaper can be used to generate a tophat beam intensity distribution through the sapphire fibers 606 by transforming a standard Gaussian laser beam 528 into a profile with uniform intensity. This can be accomplished using a diffractive optical element (DOE) that reshapes the beam's 528 wavefront. More specifically, the DOE has a complex surface pattern designed to alter the phase of the light passing through it. This uniform, flat-top profile is then directed into the sapphire fibers 606. This process ensures the beam 528 has a consistent intensity across its cross-section.

A window 516 is positioned to permit transmission of the laser beam 604 from the distal ends 610 of the optical fibers 606 toward the tissue 620. In an example, window 516 may be sapphire. The window 516 also serves as a protective barrier to prevent contamination or damage to the optical fibers 606, permitting them to be re-used after each surgery. At least one spacing element 612 is associated with the support structure 616 and houses the support structure 616 while maintaining a predetermined distance between the distal ends 610 of the optical fibers 606 and the tissue 620 during operation. According to an embodiment, the spacing element 612 ensures that the laser energy is delivered at an optimal focal distance, supporting consistent ablation depth and reducing the risk of thermal injury to surrounding tissue.

According to another aspect, the support structure 616 may be integrated with the non-sterile cartridge 640 and/or the sterile outer-housing serving as the spacing element 612. In either configuration, the support structure 616 maintains the precise spatial arrangement of the fiber tips at the distal end relative to the integrated window 516 and the tissue 620 to support uniform energy delivery across overlapping fiber outputs.

The apparatus 600 further includes a fluid delivery and localized suction system 618 associated with the least one spacing element 612 and configured to deliver a fluid to a cutting interface 622 associated with the tissue 620. The fluid delivery system 618, which may include water jet 520, can provide irrigation to the cutting site, assisting in cooling the tissue, removing debris, and maintaining a clear optical path for the laser beam 604. Fluid delivery system 618 may include an in-line pulsatile delivery system 618 associated with the support structure 616 and configured to deliver a periodic bursts of fluid to a cutting interface 622 associated with the tissue 620. The interaction of the laser source 528, optical fibers 606, support structure 616, window 516, spacing element 612, and fluid delivery system 618 enables the apparatus 600 to achieve precise, efficient, and safe resection of hard biological tissue 620 as described in the present disclosure. According to an implementation, a cooling fluid delivery system 618 may include at least one nozzle configured for pulsatile delivery of fluid to the cutting interface 622 and be supported by compressed air or an assist gas delivery system 522. For example, by applying cooling in distinct, periodic time intervals, (e.g 15, 30 or 45-seconds on), followed by a matching or non-matching off-period (rather than as a continuous flow), superior cut quality and extension of the lifespan of the equipment associated with the present disclosure can be achieved.

According to an aspect, the fluid delivery system 618 can include a localized dual-channel irrigation/suction system integrated into the spacing element 612. In an example, a 1 mm diameter longitudinal channel houses a mist nozzle configured to deliver a fine spray of saline and medical air (e.g., approximately 10-30 ml/min at 5-15 bar) locally into the ablation zone to cool tissue, reduce the risk of carbonization, and cleanse the optical path. A second longitudinal channel operates under negative pressure to evacuate residual water, debris, and plume. Pulsed or sequenced operation of fluid and air can be coordinated with laser pulses to avoid water shielding of the beam while maintaining crater cleanliness as depth increases.

In certain modes in accordance with the present disclosure, the irrigation duty cycle is configured with ON/OFF periods in the range of approximately 5-30 s (e.g., 5 s ON/5 s OFF; 10 s ON/10 s OFF) to manage crater temperature while minimizing water accumulation. Excessively high repetition rates that prevent adequate thermal recovery can be avoided to reduce the risk of carbonization; feedback-based temperature monitoring can be used to adjust duty cycles accordingly.

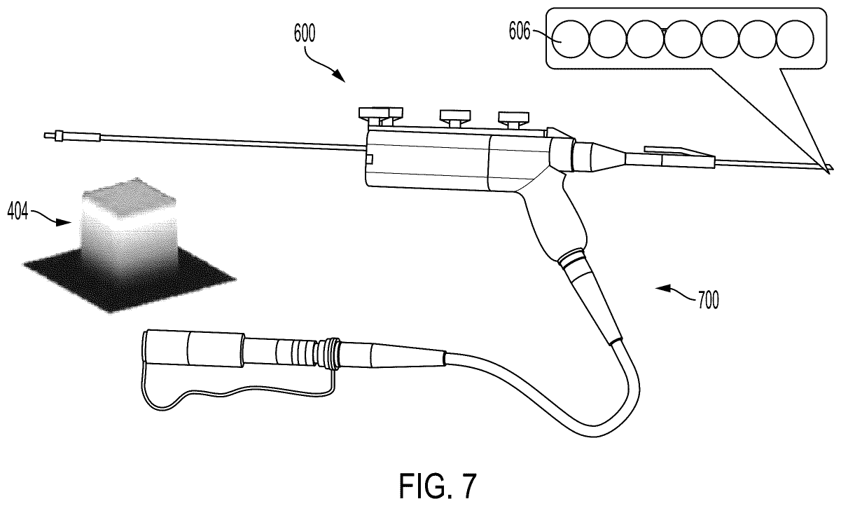

Referring to FIG. 7, a beam treatment device 700 is provided for resecting hard biological tissue 620, such as bone. The present disclosure addresses limitations of prior surgical systems that relied on mechanical tools or single-beam laser devices, which often resulted in imprecise cuts, excessive thermal damage, and inefficient tissue removal. The present disclosure includes a beam treatment device 700 that incorporates apparatus 600 configured to deliver laser energy, including a top-hat beam profile 404, to the tissue 620 in a controlled and distributed manner, thereby supporting uniform ablation, efficient resection, and improved surgical outcomes.

The beam treatment device 700 includes apparatus 600, which comprises a laser source 528 configured to emit a laser beam 604 and a plurality of optical fibers 606 arranged to deliver the laser energy to the tissue 620. The apparatus 600 is supported and positioned within the beam treatment device 700 to maintain the spatial arrangement of the optical fibers 606 and to ensure that the laser energy is directed precisely to the target tissue 620. The device 700 is configured to accommodate the apparatus 600 and facilitate its operation during resection procedures. The laser energy delivered by the apparatus 600 can include a top-hat beam profile 404, which provides a uniform intensity distribution across the ablation site, supporting consistent and controlled removal of tissue 620.

In certain use cases, performance targets include deep planar cuts (>50 mm), cut widths on the order of approximately 1.5-3.5 mm depending on oscillatory modes and fiber configuration, linear removal rates of approximately 10-30 mm/min, and volumetric removal rates ≥5 mm3/s, with total TKA femoral and tibial resection times on the order of approximately 10 minutes when operated with optimized beam shaping, fiber multiplexing, fluid management, and feedback control.

In an example, the beam treatment device 700 is operatively coupled to a laser delivery and beam shaping system 500, which can include optical pathway(s) 502 or 530 described with reference to FIG. 5. This system 500 can include an Er:YAG laser source 528, collimation and focusing lenses 506 and/or 514, one or more mirrors 508, 510, and/or 512, a window 516, and associated irrigation and monitoring components 520 and/or 522. The laser beam 604 generated and shaped by this system 500 can be directed into the plurality of optical fibers 606 of apparatus 600, which are arranged to deliver the laser energy to the tissue 620 in a controlled and distributed manner.

According to an aspect of the present disclosure, the beam treatment device 700 may further include features for stabilizing and orienting the apparatus 600 relative to the tissue 620, ensuring that the distal ends 610 of the optical fibers 606 are maintained at an optimal distance from the tissue 620 surface. The device 700 can also include integrated fluid delivery systems, such as waterjet 520 and/or fluid delivery system 618, for irrigation and cooling, as well as suction mechanisms for debris removal, such as air irrigation 522, thereby maintaining a clear optical path and reducing the risk of thermal injury. The interaction of the beam treatment device 700, apparatus 600, and the tissue 620 enables precise, efficient, and safe resection of hard biological tissue 620 as described in the present disclosure.

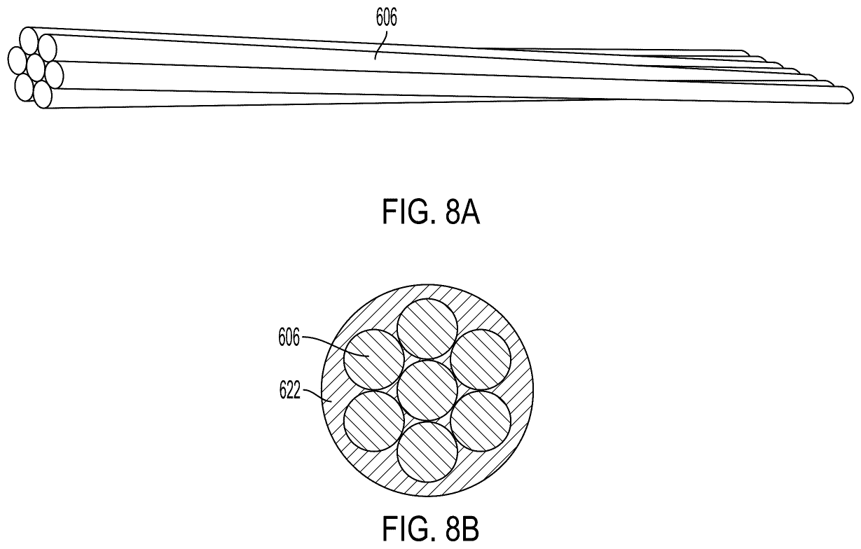

Referring to FIGS. 8A and 8B, the plurality of optical fibers 606 are illustrated as would be arranged within apparatus 600, enabling controlled, distributed, and efficient transmission of laser energy for tissue resection. More specifically, this arrangement utilizes a bundled configuration of multiple optical fibers to support uniform ablation, improved energy coupling, and enhanced operational reliability during surgical procedures. FIG. 8A shows the plurality of bare optical fibers 606 supported within the coaxial cable bundle 622, which serves as a protective housing at the proximal end 608. The coaxial cable bundle 622 maintains the alignment and organization of the optical fibers 606, shielding them from mechanical stress and environmental contaminants. Each optical fiber 606 may be a continuous sapphire fiber, typically having a diameter of approximately 400 μm and a total length of about 100 cm, with no buffer coating. In one aspect, the bundle includes seven sapphire fibers fused together at the cutting end, with approximately 15 cm of exposed fiber extending outside the bundle. The fibers may be anti-reflection (AR) coated at one end only to minimize optical losses, with the coating increasing transmission by approximately 5%. The fibers are configured to transmit laser energy at both 2.94 micron and 1.3 micron wavelengths, supporting both ablation and optical coherence tomography (OCT) analysis within the same bundle. The fibers do not require surface shaping, such as concave or convex ends, and may be optionally tapered at one or both ends to provide increased cable flexibility or to reduce damage at the air-fiber interface. For example, a sapphire fiber tapered at both ends may have a reduced diameter of 200 μm, which increases flexibility but may also increase the risk of thermal damage. Each fiber is capable of transmitting up to 1.2 J per pulse with a Gaussian beam shape.

FIG. 8B provides a front view of the plurality of optical fibers 606 within the coaxial cable bundle 622 at the proximal end 608. The bundled configuration ensures that the optical fibers 606 remain tightly packed and aligned, maximizing coupling efficiency and minimizing optical losses. The bundle may be terminated with a high-power connector, such as an SMA connector, which provides a low-loss optical interface between the laser source and the fiber bundle. The arrangement of the fibers within the coaxial cable bundle 622 allows for efficient transmission of laser energy from the source to the distal ends of the fibers, where the energy is delivered to the tissue for ablation. The bundle configuration also supports reusability of the fiber assembly, facilitates rapid connection and disconnection from the laser source, and enhances the durability and reliability of the apparatus during repeated surgical procedures.

According to an aspect of the present disclosure, the sapphire fibers 606 within the bundle are configured to transmit both the primary ablation wavelength and the OCT wavelength, enabling simultaneous tissue resection and real-time depth analysis. The absence of surface shaping and the use of AR coatings at one end of each fiber minimize energy loss and thermal damage, while the bundled arrangement ensures uniform energy delivery across the ablation site. The optional tapering of the fibers provides flexibility for different surgical scenarios, allowing the apparatus to be adapted for various anatomical sites and resection geometries. The interaction between the plurality of optical fibers 606, the coaxial cable bundle 622, and the proximal end 608 supports precise, efficient, and safe resection of biological tissue as described in the present disclosure.

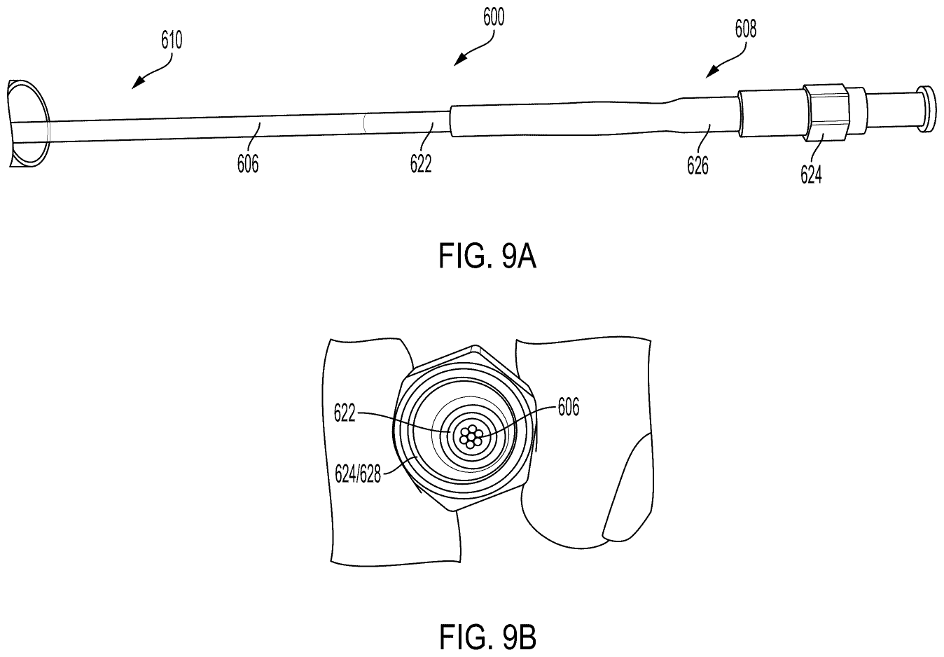

As introduced in FIG. 6 and illustrated in more detail in FIGS. 9A-9B, the present disclosure includes a proximal end 608 of each optical fiber 606 that is configured for robust and efficient coupling to the laser source 602. The proximal end 608 includes a fiber coaxial cable jacket 622, which is configured to hold the plurality of optical fibers 606 together in a bundled arrangement. The fiber bundle is, for example, approximately 45 cm in length, with the fiber coaxial cable jacket 622 extending for approximately 25 cm of that length. The fiber coaxial cable jacket 622 provides mechanical protection and organization for the optical fibers 606, ensuring that the fibers 606 remain securely aligned and are shielded from external stresses or environmental contaminants. In some embodiments and as shown in FIGS. 6 and 9A, fiber coaxial cable jacket 622 may be encompassed by jacket 626 to further ensure that the plurality of optical fibers 606 remain aligned within fiber coaxial cable jacket 622.

According to an aspect of the present disclosure, the fiber coaxial cable jacket 622 terminates at the proximal end 628 of the apparatus 600 with a high-power SMA connector 624. The SMA connector 624 is positioned at the input side of the apparatus 600 and is configured to provide a secure and low-loss optical interface between the laser source 602 and the bundled optical fibers 606. Within the SMA connector 624, the optical fibers 606 are tightly packed to maximize coupling efficiency and minimize optical losses. The SMA connector 624 can include coupling facet 628, which is provided at the interface between the SMA connector 624 and the laser source 602, ensuring precise alignment and optimal transmission of the laser beam 604 into the fiber bundle. The configuration of the fiber coaxial cable jacket 622, SMA connector 624, and coupling facet 626 supports reusability of the fiber bundle, facilitates rapid connection and disconnection from the laser source 602, and enhances the durability and reliability of the apparatus 600 during repeated surgical procedures.

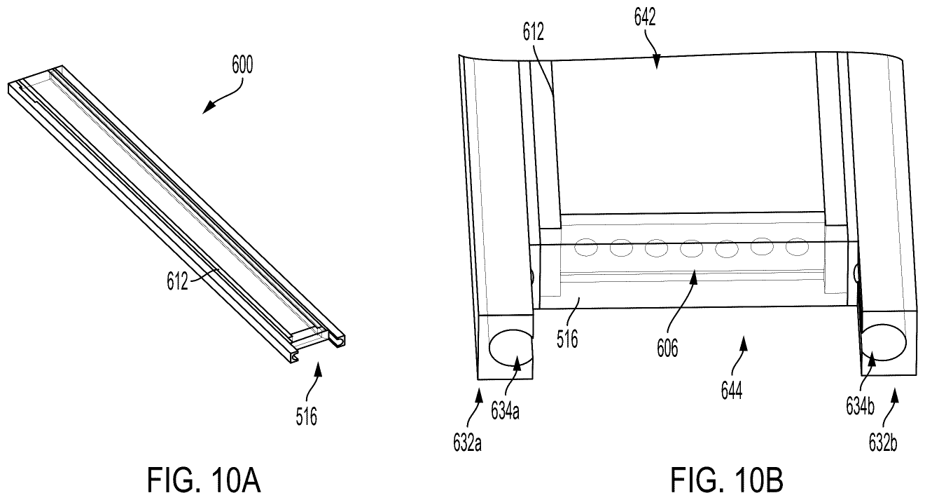

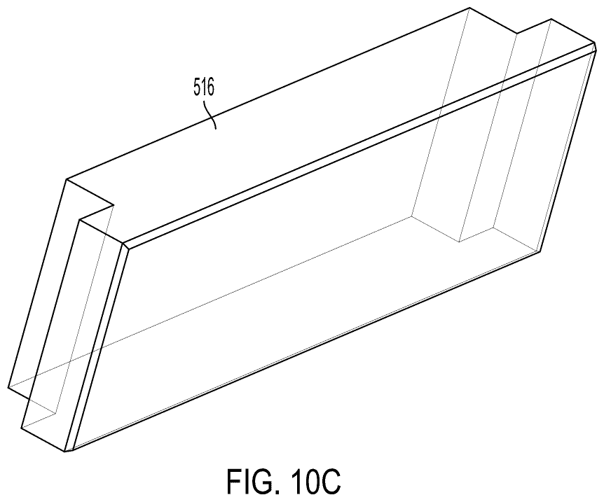

Referring to FIGS. 10A and 10B, the at least one spacing element 612 is illustrated. In an embodiment, this includes a sterile outer-housing, which maintains the distal ends 610 of the plurality of optical fibers 606 in a predetermined spatial arrangement relative to the tissue. This can also the support a homogenous intensity distribution at the ablation site and enable precise, efficient, and reproducible tissue resection. More specifically, the sterile outer-housing, serving as the at least one spacing element 612, may be formed from medical-grade 6061 aluminum alloy and may be either disposable or re-usable. The outer-housing has dimensions of approximately 10 mm by 2 mm by 80 mm and is designed to enclose and support the plurality of optical fibers 606. The outer-housing includes extruded projections 632a and 632b, which extend approximately 2 mm to 2.5 mm from the distal end. In an embodiment, these extruded projections 632a and 632b function to control the distance between the bone surface 620 and the distal ends 610 of the optical fibers 606 during cutting, ensuring that laser energy is delivered at an optimal focal distance for efficient and consistent ablation. The support structure 616, integrated within the outer-housing, maintains the spatial arrangement of the fibers 606 and can be observed through an integrated window 516. As illustrated in FIG. 10C, window 516, with dimensions of approximately 7 mm by 2 mm by 1 mm in an example, provides a clear view of the fiber arrangement and the ablation site, while also serving as a protective barrier against contamination and mechanical damage.

According to another aspect of the present disclosure, the sterile outer-housing further includes two equidistant channels 634a and 634b within extruded projections 632a and 632b, respectively. In an example, each includes a diameter of approximately 1.5 mm, and are configured for circulating mist and for plume suction as irrigation and/or air systems 520, 522, and 618. These channels 634a and 634b enable the delivery of a fine mist or waterjet to the ablation site, which serves to cool the tissue, minimize thermal injury, and facilitate the removal of bone debris generated during resection. The suction channel (e.g., channel 634a) operates in conjunction with the mist generator (e.g., channel 634b) to evacuate excess fluid and ablation byproducts, maintaining a clear optical path for the laser energy and supporting uninterrupted operation. For example, the suction channel 634a may be configured to evacuate excess fluid and ablation byproducts from the cutting interface 622 in order to maintain the optimum cutting efficiency achieved at the surface with increasing depth. A central port 642 is provided for inserting the cartridge 640, which contains and organizes the plurality of optical fibers 606. The support structure 616, which may be part of the cartridge 640 and/or the sterile outer-housing, maintains the spatial arrangement of the fiber tips at the distal end, ensuring precise delivery of laser energy through the window 516. In an example, central port 642 includes dimensions of approximately 5.9 mm by 1.5 mm by 80 mm and/or includes a modular design to allow for rapid assembly and disassembly, supporting both single-use and reusable workflows.

The interaction between the at least one spacing element 612, the support structure 616, the distal ends 610 of the optical fibers 606, the integrated window 516, the extruded projections 632a and 632b, the mist and suction channels 634a and 634b, cartridge 640, and the central port 642 enables the present disclosure to achieve a high-intensity region 644 at the ablation site with precise control over energy delivery and tissue interaction. The homogenous intensity distribution achieved by the overlapping regions of the laser fibers 606, combined with efficient cooling and debris management, supports safe, effective, and reproducible resection of biological tissue.

Referring to FIGS. 11-12E, the present disclosure includes a cartridge 640 and a support structure 616 configured to enable precise, efficient, and reliable delivery of laser energy for resecting biological tissue. Prior to the present disclosure, surgical systems often suffered from imprecise fiber alignment, inconsistent energy delivery, and challenges in maintaining sterility while enabling reuse of costly optical components. The present disclosure overcomes these limitations by including a modular cartridge 640 and support structure arrangement 616 that enables accurate positioning of multiple optical fibers 606, facilitates rapid assembly and disassembly, and supports both single-use and reusable workflows while maintaining the integrity and alignment of the fiber bundle 606.

In an example, the cartridge 640 is non-sterile, and is constructed from a material such as PTFE or Delrin and has dimensions of approximately 6 mm by 1.4 mm by 80 mm. At a distal end 648b, the cartridge 640 may include longitudinal channels 646 configured to house optical fibers 606, each having a diameter of approximately 0.5 mm, arranged in a linear configuration and equally spaced 0.8 mm apart. For example, this arrangement ensures that each optical fiber 606 is held in a precise and parallel orientation, supporting uniform energy delivery and minimizing optical losses. In an embodiment, the cartridge 640 is further configured to receive approximately 20 cm of free, bare optical fibers 606, which are inserted into the longitudinal channels 646 without coatings on the fiber tips. For example, this configuration allows the distal ends of the fibers 606 to be positioned accurately at the ablation interface, supporting controlled and distributed emission of laser energy associated with a beam 604.

In an embodiment of the present disclosure, the sterile outer-housing (spacing element 612) can be formed from medical-grade 6061 aluminum alloy; in certain aspects, alternative rigid materials (e.g., 17-4 stainless steel) may be used where higher stiffness or dimensional stability is preferred to immobilize the fibers and maintain precise alignment of the end facet relative to the integrated window 516. The two-piece configuration (sterile outer-housing and non-sterile cartridge 640) facilitates sterile handling and post-use disposal of the housing while preserving the reusable fiber assembly.

According to another aspect, at the proximal end 648a of the cartridge 640, a connector 650 is provided, which may include locator pins 652a and/or 652b to ensure precise and predictive insertion within the cannulation of the spacing element 612, which can include an outer sterile housing. The connector 650 supports robust mechanical and optical coupling to the laser source 528, maintaining the alignment of the fibers 606 and facilitating rapid connection and disconnection. The use of locator pins 652a and/or 652b and a defined connector 650 geometry ensures that the cartridge 640 can be inserted into the sterile housing in a repeatable and reliable manner, reducing the risk of misalignment or damage to the fibers 606 during assembly.

FIGS. 12A-12E include images 1200, 1210, 1220, 1230, and 1240, respectively, detailing various views of the dynamic between support structure 616 and cartridge 640. These images provide different perspectives on how the support structure 616 encompasses and interacts with the cartridge 640 to maintain the spatial arrangement of the optical fibers 606 at the distal end 610 of the apparatus 600. For example, connector 650 may be further configured to interface with support structure 616, as shown in FIGS. 12A-12E. According to an aspect, this may further secure apparatus 600 within beam treatment device 700. For reference, an optical fiber 606 is included and shown in these images to demonstrate the precise positioning and alignment achieved by the interaction of the support structure 616 and cartridge 640. The support structure 616 provides mechanical stability and accurate orientation of the fiber tips relative to the ablation interface, ensuring that laser energy is delivered in a controlled and distributed manner. The support structure 616 interacts with the cartridge 640 to secure the fibers in the desired configuration and interfaces with the outer sterile housing to maintain sterility and facilitate integration with other system components.

According to an aspect, the interaction between the cartridge 640, the support structure 616, the optical fibers 606, and the spacing element 612 enables the present disclosure to achieve a high degree of modularity, reusability, and precision in laser energy delivery. The cartridge 640 and support structure 616 support the maintenance of a sterile field by allowing the reusable fiber assembly of apparatus 600 to remain isolated from the surgical environment, while the spacing element 612 provides the necessary interface with the tissue 620 and associated fluid management systems 520, 522, and/or 618. This configuration supports safe, effective, and reproducible resection of biological tissue 620 as described in the present disclosure.

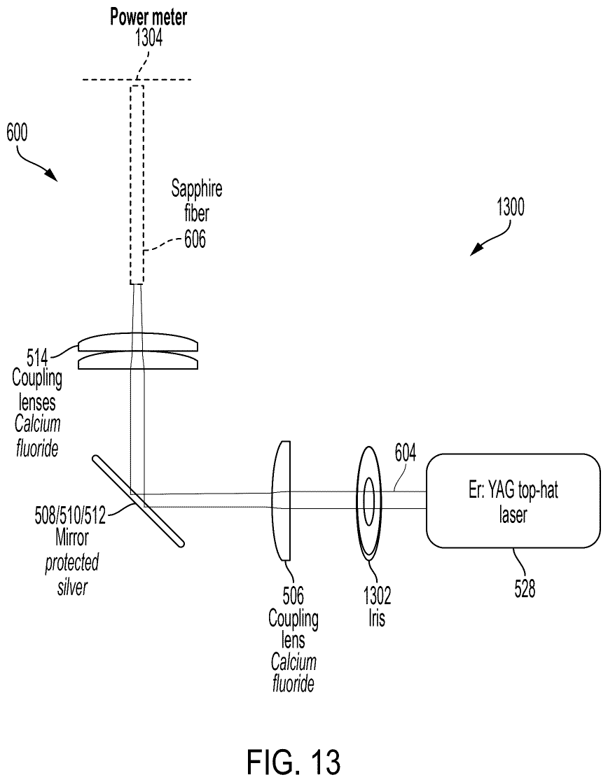

Referring to FIG. 13, the present disclosure includes a top-hat ER laser optical system 1300 configured to deliver controlled, high-efficiency laser energy for resecting biological tissue 620. The top-hat ER laser optical system 1300 includes a laser source 528, which may be an Er:YAG laser operating at a wavelength of approximately 2.94 μm and configured to deliver an average power of up to 30 W. According to an aspect, the laser source 528 emits a laser beam 604, which is directed through an iris 1302 to control the beam diameter and intensity profile. Iris 1302 may include an adjustable, high-durability aperture mechanism with precision control, mounting features, and coatings to optimize laser beam 604 shaping, safety, and system integration for surgical laser applications associated with system 1300. The beam 604 then passes through a coupling lens 506, which may be fabricated from calcium fluoride, to collimate and focus the laser energy. The collimated beam 604 is subsequently directed by one or more mirrors 508, 510, and/or 512, which may be constructed from protected silver to maximize reflectivity and minimize energy loss. After reflection, the beam 604 passes through a second coupling lens 514, also made of calcium fluoride, to further refine the beam 604 profile and ensure optimal coupling into the fiber delivery system of apparatus 600.

According to another aspect of the present disclosure, the laser beam 604 is then delivered into apparatus 600, which includes a fiber bundle 606. In an example, the fiber bundle 606 may be composed of seven solid sapphire fibers, each having a diameter of approximately 425 μm and no buffer coating, arranged in a multiplexed configuration with an overall transmission efficiency of between 70 and 80% with a tophat intensity distribution that maximizes energy delivery and cutting efficiency. The fiber bundle 606 may have a working length of 30 to 50 cm, with the cutting end shaped as a sphere and the multiplexed end arranged in a hexagonal pattern to improve fiber coupling. According to an aspect, the system 1300 is configured to operate at a repetition rate of less than 20 Hz to avoid premature degradation of the fibers. The fiber bundle 606 is designed to deliver a non-Gaussian, line beam shape, supporting a top-hat intensity profile for uniform ablation. Further, in an example, the system 1300 is capable of delivering up to 430 mJ per fiber, with a worst-case scenario of 350 mJ per fiber and a total pulse energy of up to 3000 mJ, enabling efficient resection of tissue at various depths and widths.

As previously discussed, the apparatus 600 is configured to maintain a cut distance from the hard tissue 620, or bone, of approximately 2 mm, supporting a focused beam approach that contrasts with prior systems requiring much greater distances between the fiber tip and the tissue. More specifically, the system 1300 is capable of achieving cuts 2 mm deep by 3.5 mm wide in approximately 10 seconds, with water cooling and evacuation cycles of 5 seconds. For deeper cuts, such as 20 mm or 50 mm, the system 1300 can complete the resection in approximately 2.5 minutes and 12.5 minutes, respectively, including appropriate cooling cycles. The modular design of apparatus 600, as previously discussed, allows for the addition of more laser fibers 606 to further increase cutting speed, provided that additional laser sources 528 and fibers 606 are incorporated.

According to another aspect of the present disclosure, power meter output 1304 is included to monitor the delivered energy and ensure consistent system 1300 performance. The system 1300 supports power scaling, with different current (A) levels resulting in different pulse energy (mJ) outputs, allowing for precise adjustment of the delivered laser energy based on procedural requirements. In an aspect, Table 1 illustrates example power meter output 1304 associations:

TABLE 1

| Current | Pulse Energy |
|---|---|
| (A) | (mJ) |
| 50 | 7.82 |
| 60 | 25.39 |
| 70 | 45.8 |
| 80 | 62.90 |
| 90 | 76.2 |
| 100 | 94.8 |
| 110 | 101.4 |
| 120 | 109.8 |
| 130 | 117.7 |
| 140 | Fibre tip burned |
| 150 | |

The collimation of the beam 604 throughout the optical pathway is configured to prevent thermal damage to the fibers 606 and surrounding tissue in proximity to hard tissue 620, supporting safe and effective operation. The interaction of the laser source 528, iris 1302, coupling lenses 506 and 514, mirrors 508, 510, and 512, apparatus 600, fiber bundle 606, and power meter 1304 enables the present disclosure to achieve precise, efficient, and reproducible resection of biological tissue 620.

According to another aspect of the present disclosure, representative power-scaling protocol may include monitoring fiber-coupled pulse energy as a function of laser drive current with the iris 1302 set to define the beam diameter at the coupling lens 506, thereby optimizing coupling without exceeding fiber damage thresholds. The system 1300 can be adjusted (e.g., via iris 1302 and lens positions 506, 514) to preserve a top-hat intensity at the coupling plane, thereby maintaining beam quality through the fiber bundle 606. Inline power meter output 1304 supports closed-loop verification of delivered energy.

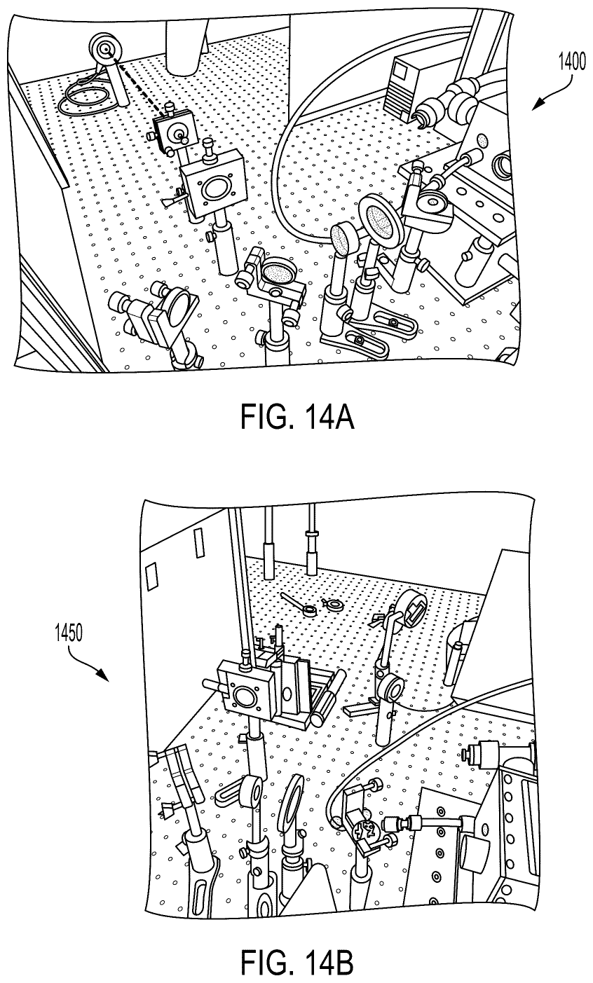

Referring further to FIGS. 14A and 14B, the present disclosure includes images 1400 and 1450, respectively, which provide additional detail regarding the configuration and operation of the laser optical system 1300. Image 1400 depicts the laser optical system 1300 without the optical fibers installed, thereby highlighting the arrangement and alignment of the upstream optical components, including the laser source 528, iris 1302, coupling lenses 506 and 514, and mirrors 508, 510, and 512. This view demonstrates the unobstructed optical pathway and the manner in which the laser beam 604 is shaped, collimated, and directed prior to entering the fiber delivery system of apparatus 600.

Image 1450, in contrast, shows the laser optical system 1300 with the optical fibers 606 installed. In this configuration, the interaction between the shaped and collimated laser beam 604 and the fiber bundle 606 is clearly demonstrated. The presence of the optical fibers 606 in image 1450 illustrates the coupling of the laser energy 604 into the multiplexed fiber arrangement of 606, as described in connection with apparatus 600. This view further emphasizes the integration of the fiber bundle 606 with the downstream optical pathway and the manner in which the system is configured to deliver uniform, high-efficiency laser energy for tissue resection.

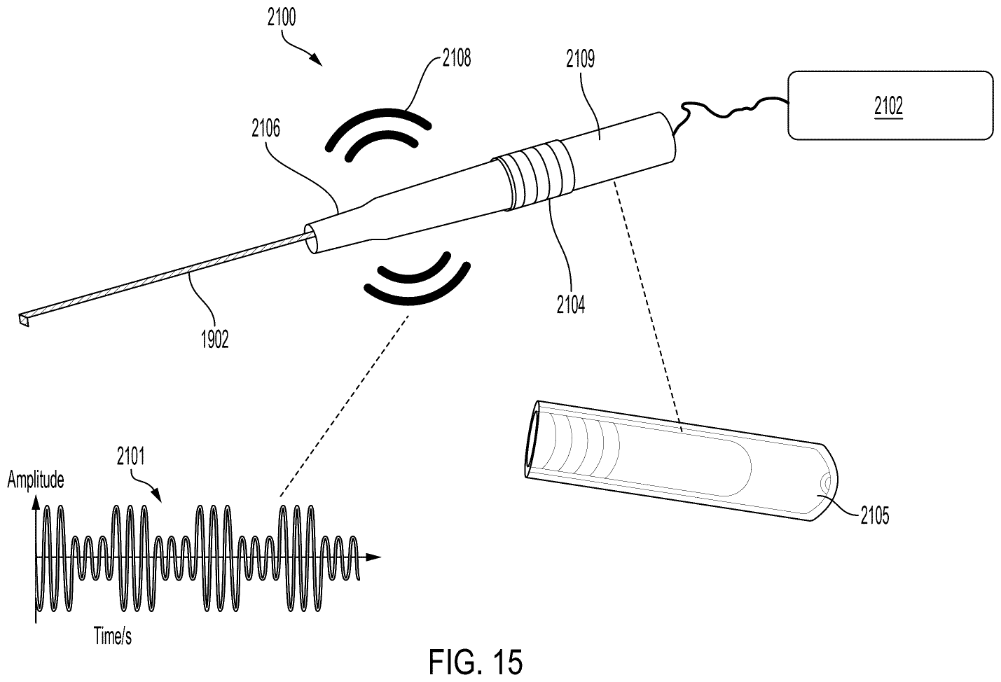

Referring to FIG. 15, the present disclosure includes a piezoelectric actuation system 2100 that may be incorporated into apparatus 600, beam treatment device 700, and/or system 1300 to provide optional vibration-assisted laser cutting. Prior to the present disclosure, laser resection systems often faced limitations in cut width, efficiency, and the ability to safely resect bone 620 adjacent to sensitive soft tissue structures. The present disclosure overcomes these limitations by including a piezoelectric actuation system 2100 that enables programmable vibration of the end effector 1902, thereby enhancing the laser cutting process through both linear oscillation and ultrasonic vibration.

In an aspect, the piezoelectric actuation system 2100 is configured to operate at a device power of approximately 5 to 10 W. The actuator 2100 is capable of vibrating the end effector 1902, supporting laser cutting in a selected direction. The vibration frequency 2101 may be programmable and is generated by applying an electric current from a generator 2102 to a set of piezoceramic rings 2104. The application of current causes the piezoceramic rings 2104 to deform, resulting in linear movement. This movement produces vibration in a transducer 2105 and/or amplifier 2106, which generates an ultrasound output. The ultrasound output may be further enhanced by a resonator 2109. The resulting mechanical waves 2106 are transmitted to the end effector 1902 of apparatus 600, beam treatment device 700, and/or system 1300, where the longitudinal movement produced by the vibrations can increase the effective cut width of the laser, for example, from 7 mm to over 20 mm.

According to another aspect of the present disclosure, the system 2100 supports a “laser sweeping” mode, in which linear oscillation of the end effector 1902 is provided at frequencies between approximately 50 and 300 Hz. This mode extends the cut width of the laser, with the width being controlled by the harmonics and amplitude of the oscillation. The laser sweeping mode also prevents the end effector 1902, or laser resection blade 1902, associated with apparatus 600 from jamming as it is plunged into bone 620, thereby reducing the risk of bone inter-digitation during cutting and supporting efficient bone removal. For more delicate procedures, the system 2100 supports an “ultrasonic” mode, in which the end effector 1902 is vibrated at higher frequencies, such as 25 to 30 kHz, which is near the relaxation frequency of bone 620. This ultrasonic mode enables mechanical resection of bone 620 in corners or regions adjacent to critical structures, such as nerves, ligaments, and blood vessels, or deep cutting in the proximal tibia, which is difficult to visualize, allowing for precise removal of bone 620 while minimizing risk to surrounding soft tissue.

In addition to linear oscillation (“laser sweeping”) in the range of approximately 50-300 Hz to extend cut width (controlled by harmonics and amplitude) and to prevent jamming as the blade advances into bone 620, the system 2100 supports operation in an “ultrasonic” mode (e.g., approximately 25-30 kHz) to facilitate mechanical resection in corners or regions adjacent to critical structures (e.g., nerves, ligaments, blood vessels). Intermittent modulation between high- and low-intensity vibrations can promote soft tissue recovery and reduce collateral effects. For tissue-selective procedures (e.g., chondroplasty), operation at frequencies above approximately 50 kHz can be used to tune depth-of-cut for specific soft tissues.

The system 2100 may be intermittently modulated between high and low intensity vibrations, which enables recovery of soft tissue and further reduces the risk of collateral damage. For tissue-specific procedures, such as chondroplasty, the system 2100 may be operated at frequencies greater than 50 kHz, allowing for selective cutting of soft tissue, including nerves and vessels, and enabling the depth of cut to be tuned according to the tissue type. The vibrational aid provided by the piezoelectric actuation system 2100 may be included in either laser-in or laser-out configurations, supporting a range of surgical applications and enhancing the versatility and safety of the apparatus 600.

The present disclosure also includes optional multi-modal feedback systems. A temperature sensor (e.g., thermocouple) can be positioned within apparatus 600 or beam treatment device 700 for safety monitoring, with an over-temperature setpoint configured to shut down the laser source 528. In an example, the sensor may be a non-contact sensor. According to an implementation, the sensor may use light to create high-resolution, cross-sectional images of the bone 620, allowing for precise monitoring of the ablation depth as the process occurs. The fiber bundle 606 can optionally include a dedicated OCT fiber configured for 1.3-1.5 μm operation, providing real-time crater depth measurement (e.g., “7th degree of freedom”) to complement 6DOF positional tracking. Additional modalities such as optoacoustic/photoacoustic imaging and/or laser-induced breakdown spectroscopy (LIBS) can be included for tissue differentiation and process monitoring. The cutting action of the handheld laser tool can be supported by cutting guides for additional safety control or guide-free reliant under control by the 6DOF optical tracking-based navigation system.

While the apparatus 600 is described for planar cuts with multiplexed fibers arranged in a linear array at the distal end, alternative arrangements are contemplated. The fibers can be rearranged for circular, arcuate, spherical, or custom distributions to produce holes, curved planes, or resurfacing patterns. In certain aspects, one or more flexible fibers can be used for non-line-of-sight access (e.g., in hip, spine, or oral/maxillofacial procedures), with a diagnostic fiber (e.g., OCT wavelength) and a cutting fiber (e.g., 2.94 μm) housed within a small-diameter tube (e.g., approximately 1 mm outer diameter) to reach confined spaces. The usable fiber length can be selected to balance maneuverability and transmission efficiency; in certain aspects, lengths on the order of approximately 1 m are preferred for coupling to a proximal laser box while maintaining acceptable loss.

As previously introduced, the systems of the present disclosure can be integrated with a robotic guidance platform (e.g., CASS 100) for either guide-assisted or guide-free operation. In certain aspects, guide-free cutting can be supported by combining 6DOF tracking with inline OCT depth sensing to constrain cut progression to the planned 3D shape model, with automatic cessation of laser output upon reaching a programmed depth boundary. For handheld use, the spacing element 612 feet maintain the fiber-to-bone distance while optional oscillatory motion spreads energy deposition to manage local heating and promote efficient depth advancement.

In some aspects of the present disclosure, waterjet-guided laser (WJGL) machining can be used to increase working distance while maintaining beam confinement via total internal reflection within a laminar water column. Where used, coolant delivery may be interleaved with laser pulses to avoid water shielding and maintain a clean path at depth. Pressurized air delivery can be used in parallel to clear debris and droplets from the focusing window and crater, with suction used to control accumulation as depth increases.

According to another aspect of the present disclosure, in light of prior art reports of prolonged CO2 laser osteotomy times (e.g., approximately 69 minutes for a five-cut femoral TKA resection), the present disclosure targets total TKA femoral and tibial cut times on the order of approximately 10 minutes. This target may be achieved by combining beam shaping for efficient energy use, multiplexed fibers operated at a near-surface standoff, localized mist/suction duty cycles to maintain a clear optical path at depth, feedback control (e.g., OCT), and optional oscillatory assistance to widen effective cut width.

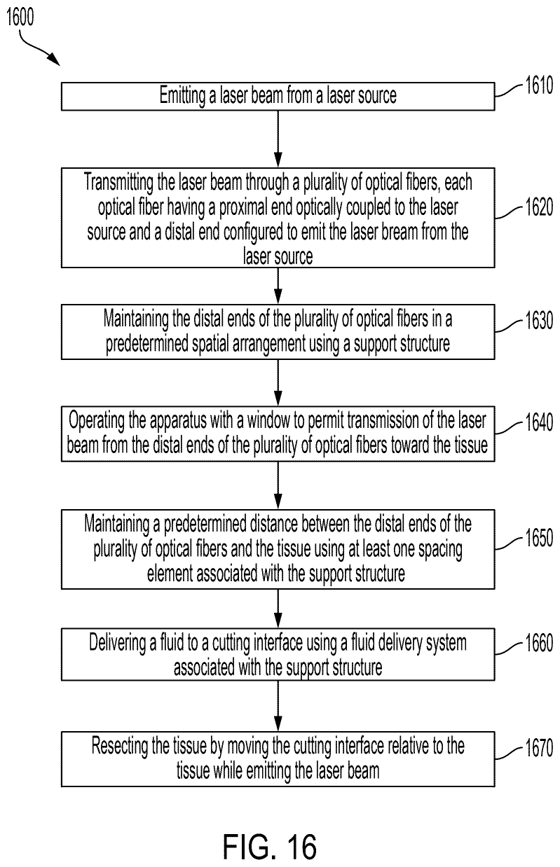

FIG. 16 is a flowchart of an example method 1600 for resecting biological tissue, such as bone 620. At step 1610, emitting a laser beam from a laser source 528, wherein the laser source is configured to generate a beam with a predetermined wavelength, pulse duration, and intensity suitable for ablation of hard tissue. For example, the laser source 528 may be an Er:YAG laser operating at a wavelength of 2.94 μm, with a pulse duration of 250 μs and a pulse energy of 400 mJ per pulse. The laser may be operated in a pulsed mode at a repetition rate of 10 Hz to 50 Hz, and may be controlled by a foot pedal or robotic controller 700.

Optionally, shaping the emitted laser beam to have a top-hat or other non-Gaussian intensity profile using one or more optical elements or diffractive optics 506, 514, to improve ablation efficiency and cut quality. For example, a diffractive optical element or beam-shaping lens 506 may be used to convert a Gaussian beam into a flat-top (top-hat) profile, resulting in a more uniform ablation crater and straighter cut walls. The shaped beam may be verified using a beam profiler 1304 prior to delivery to the tissue.

At step 1620, transmitting the laser beam through a plurality of optical fibers 606, each optical fiber having a proximal end optically coupled to the laser source 528 and a distal end configured to emit the laser beam from the laser source. For example, the system may use a bundle of seven sapphire fibers 606, each 400 μm in diameter and 1 meter in length, arranged in a linear or hexagonal array. The fibers 606 may be AR-coated at one or both ends to minimize transmission losses.

At step 1630, maintaining the distal ends of the plurality of optical fibers 606 in a predetermined spatial arrangement using a support structure 616, such that the emitted beams overlap to form a line-shaped or otherwise engineered ablation profile. For example, the support structure 616 may be a rigid cartridge or blade made of medical-grade aluminum or stainless steel, with precision-machined holes to hold each fiber 606 in a fixed position. The fibers may be spaced 0.8 mm apart to create a continuous line of laser energy at the cutting interface.

At step 1640, operating the apparatus 600 with a window 516 disposed at a distal region of the support structure 616 and configured to permit transmission of the laser beam from the distal ends of the plurality of optical fibers 606 toward the tissue 620, the window 516 being a protective, transparent element (e.g., sapphire or glass) that maintains optical clarity and reduces contamination. For example, a 7 mm×2 mm×1 mm sapphire window 516 may be fixed at the distal end of the blade using medical-grade adhesive, providing a sterile barrier and protecting the fiber tips from blood and debris.

At step 1650, maintaining a predetermined distance between the distal ends of the plurality of optical fibers 606 and the tissue using at least one spacing element 612 associated with the support structure 616, such as extruded feet or a standoff, to ensure optimal ablation conditions and safety. For example, the blade may include two 2.5 mm deep feet 612 at the distal end, which rest on the bone surface and maintain a 2 mm standoff between the fiber tips and the tissue, ensuring consistent ablation and preventing accidental contact.

At step 1660, delivering a fluid to a cutting interface using a fluid delivery system 618 associated with the support structure 616, the fluid comprising a mist, spray, or waterjet, optionally delivered in a pulsed or sequenced manner, and optionally accompanied by suction or pressurized air to remove debris and maintain a clear optical path. For example, a 1 mm diameter channel 618 may deliver a fine mist of saline and medical air at 15 bar pressure and 20 ml/min flow rate directly to the ablation site. A second channel 618 may provide suction at −100 mmHg to evacuate excess fluid and bone debris. The fluid delivery may be pulsed (e.g., 0.5 s ON, 0.5 s OFF) in synchronization with laser pulses to avoid water shielding.

Optionally, coordinating the timing of fluid delivery with laser emission (e.g., interleaved ON/OFF cycles) to maximize ablation efficiency and minimize thermal damage or carbonization. For example, the control system 700 may deliver mist only during the OFF period between laser pulses, or may use a feedback loop to adjust mist delivery based on real-time temperature measurements at the cutting interface.

Optionally, evacuating excess fluid and ablation byproducts from the cutting interface using a suction system 618 integrated with the support structure 616. For example, a negative pressure channel 618 may continuously remove water, vapor, and bone plume from the ablation crater, preventing accumulation and maintaining a clear optical path for the laser.

At step 1670, resecting the tissue by moving the cutting interface relative to the tissue while emitting the laser beam, wherein the movement may be performed robotically, manually, or with oscillatory/vibrational assistance to increase cut width and efficiency. For example, the blade may be swept linearly across the bone surface at a speed of 10 mm/min, or may be oscillated at 100 Hz using a piezoelectric actuator 2100 to widen the effective cut. The movement may be guided by a robotic arm with 6DOF tracking 700, or by a surgeon using a handpiece with built-in tracking markers.

Optionally, monitoring one or more process parameters in real time, including temperature at the cutting interface, ablation depth (e.g., using OCT fiber 606 or optoacoustic feedback), and tissue differentiation (e.g., using LIBS or photoacoustic imaging sensors). For example, a thermocouple embedded in the blade 600 may provide real-time temperature data to the control unit, which can shut off the laser if a threshold is exceeded. An OCT fiber 606 may provide depth measurements with micron resolution, and a LIBS sensor may detect the presence of soft tissue to prevent accidental injury.

Optionally, automatically adjusting laser parameters, fluid delivery, or movement based on feedback signals to optimize resection performance and ensure safety (e.g., automatic shutoff upon reaching a programmed depth or detecting vital tissue). For example, the system 700 may reduce laser power or increase mist delivery if temperature rises, or may stop the procedure if the planned resection depth is reached or if non-bone tissue is detected.

Optionally, recording or displaying process data for intraoperative guidance, quality assurance, or post-operative analysis. For example, the system 700 may display a real-time ablation map on a surgical navigation screen, log all process parameters for later review, and provide alerts or guidance to the operator as needed.

While various illustrative embodiments incorporating the principles of the present teachings have been disclosed, the present teachings are not limited to the disclosed embodiments. Instead, this application is intended to cover any variations, uses, or adaptations of the present teachings and use its general principles. Further, this application is intended to cover such departures from the present disclosure as come within known or customary practice in the art to which these teachings pertain.

In the above detailed description, reference is made to the accompanying drawings, which form a part hereof. In the drawings, similar symbols typically identify similar components, unless context dictates otherwise. The illustrative embodiments described in the present disclosure are not meant to be limiting. Other embodiments may be used, and other changes may be made, without departing from the spirit or scope of the subject matter presented herein. It will be readily understood that various features of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various features. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices also can “consist essentially of” or “consist of” the various components and steps, and such terminology should be interpreted as defining essentially closed-member groups.

In addition, even if a specific number is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, sample embodiments, or drawings, should be understood to contemplate the possibilities of including one of the terms, either of the terms, or both terms. For example, the phrase “A or B” will be understood to include the possibilities of “A” or “B” or “A and B.”

In addition, where features of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

The term “about,” as used herein, refers to variations in a numerical quantity that can occur, for example, through measuring or handling procedures in the real world; through inadvertent error in these procedures; through differences in the manufacture, source, or purity of compositions or reagents; and the like. Typically, the term “about” as used herein means greater or lesser than the value or range of values stated by 1/10 of the stated values, e.g., ±10%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the present disclosure include equivalents to the recited values, e.g., variations in the numerical quantity of such values that can occur, but would be recognized to be equivalents by a person skilled in the art.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. An apparatus for resecting hard biological tissue, comprising:
a laser source configured to emit a laser beam;
a plurality of optical fibers, each optical fiber having a proximal end optically coupled to the laser source and a distal end configured to emit the laser beam from the laser source;
a support structure configured to maintain the distal ends of the plurality of optical fibers in a predetermined spatial arrangement;
a window positioned configured to permit transmission of the laser beam from the distal ends of the plurality of optical fibers toward the tissue;
at least one spacing element associated with the support structure and configured to maintain a predetermined distance between the distal ends of the plurality of optical fibers and the tissue during operation; and
a fluid delivery system associated with the support structure and configured to deliver a fluid to a cutting interface associated with the tissue.

2. The apparatus of claim 1, wherein:
the plurality of optical fibers are arranged in a linear or hexagonal array at the distal ends of the plurality of optical fibers.

3. The apparatus of claim 1, wherein:
the laser source is further configured to emit the laser beam having a substantially uniform intensity profile at the distal ends of the plurality of optical fibers.

4. The apparatus of claim 1, wherein:
the window comprises a material selected from the group consisting of sapphire and glass.

5. The apparatus of claim 1, wherein:
the at least one spacing element comprises one or more projections extending from the support structure and configured to contact the tissue and to maintain the predetermined distance.

6. The apparatus of claim 1, wherein:
the fluid delivery system comprises at least one nozzle configured to deliver a mist of fluid to the cutting interface.

7. The apparatus of claim 6, wherein:
the fluid delivery system further comprises a suction channel configured to evacuate excess fluid and ablation byproducts from the cutting interface.

8. The apparatus of claim 1, further comprising:
a sensor associated with the support structure and configured to provide real-time feedback regarding at least one of temperature, ablation depth, or tissue differentiation at the cutting interface.

9. The apparatus of claim 8, wherein:
the sensor comprises an optical coherence tomography (OCT) sensor configured to measure ablation depth.

10. The apparatus of claim 8, further comprising:
one or more processors configured to receive feedback from the sensor and to control at least one of the laser source, the fluid delivery system, or the support structure based on the feedback.

11. The apparatus of claim 1, wherein:
the support structure comprises a disposable outer portion and a reusable inner portion containing the plurality of optical fibers.

12. The apparatus of claim 1, wherein:
the apparatus is configured for use with a robotic guidance system or a hand-held operation mode.

13. The apparatus of claim 1, wherein:
the laser source is configured to emit at a wavelength between 2.7 and 3.1 microns.

14. The apparatus of claim 1, further comprising:
an actuator configured to oscillate the support structure to increase the effective width of the resection at the cutting interface.

15. The apparatus of claim 14, wherein:
the actuator comprises a piezoelectric transducer configured to impart linear or ultrasonic oscillation to the support structure.

16. An apparatus for resecting hard biological tissue, comprising:
a laser source configured to emit a laser beam;
a plurality of optical fibers, each optical fiber having a proximal end optically coupled to the laser source and a distal end configured to emit the laser beam from the laser source;
a support structure configured to maintain the distal ends of the plurality of optical fibers in a predetermined spatial arrangement;
a window positioned configured to permit transmission of the laser beam from the distal ends of the plurality of optical fibers toward the tissue;
at least one spacing element associated with the support structure and configured to maintain a predetermined distance between the distal ends of the plurality of optical fibers and the tissue during operation;
a fluid delivery system associated with the support structure and configured to deliver a fluid to a cutting interface associated with the tissue; and
a suction channel integrated with the support structure and configured to evacuate excess fluid and ablation byproducts from the cutting interface.

17. The apparatus of claim 16, further comprising:
a sensor comprising an optical coherence tomography (OCT) sensor configured to measure ablation depth at the cutting interface.

18. A method for resecting hard biological tissue, comprising:
emitting a laser beam from a laser source;
transmitting the laser beam through a plurality of optical fibers, each optical fiber having a proximal end optically coupled to the laser source and a distal end configured to emit the laser beam from the laser source;
maintaining the distal ends of the plurality of optical fibers in a predetermined spatial arrangement using a support structure;
positioning a window to permit transmission of the laser beam from the distal ends of the plurality of optical fibers toward the tissue;
maintaining a predetermined distance between the distal ends of the plurality of optical fibers and the tissue using at least one spacing element associated with the support structure;
delivering a fluid to a cutting interface using a fluid delivery system associated with the support structure; and
resecting the tissue by moving the cutting interface relative to the tissue while emitting the laser beam.

19. The method of claim 18, further comprising:
arranging the plurality of optical fibers in a linear or hexagonal array at the distal ends of the plurality of optical fibers.

20. The method of claim 18, further comprising:
evacuating excess fluid and ablation byproducts from the cutting interface using a suction channel integrated with the support structure.
